(19) United States
(12) Patent Application Publication
(10) Pub. No.: US 2025/0282864 A1
(43) Pub. Date: Sep. 11, 2025

(54) SUBCUTANEOUS ADMINISTRATION OF CD19-BINDING T CELL ENGAGERS

(71) Applicant: AMGEN RESEARCH (MUNICH) GMBH, Munich (DE)

(72) Inventor: Gerhard Zugmaier, Munich (DE)

(21) Appl. No.: 18/704,120

(22) PCT Filed: Oct. 14, 2022
(86) PCT No.: PCT/EP2022/078638

§ 371 Date: Apr. 24, 2024

Related U.S. Application Data
(60) Provisional application No. 63/256,056, filed on Oct. 15, 2021.
(60) Provisional application No. 63/281,992, filed on Nov. 22, 2021.

(51) Int. Cl. C07K 16/28; A61K 9/00; A61K 39/00; A61K 40/33; A61K 40/42; A61K 47/02; A61K 47/18; A61K 47/26; A61K 47/40; A61P 35/02

(52) U.S. Cl. CPC C07K 16/2803; A61K 9/0019; A61K 40/33; A61K 40/4211; A61K 47/02; A61K 47/183; A61K 47/26; A61K 47/40; A61P 35/02; C07K 16/2809; A61K 2039/505; A61K 2039/54; A61K 2039/545; A61K 2239/31; A61K 2239/38; A61K 2239/48; C07K 2317/622; C07K 2319/31

(57) ABSTRACT

The present invention provides bispecific antibody constructs of a specific Fc modality characterized by comprising a first domain binding to a target cell surface antigen, a second domain binding to an extracellular epitope of the human and/or the Macaca CD3 chain and a third domain, which is the specific Fc modality. Moreover, the invention provides a polynucleotide, encoding the antibody construct, a vector comprising this polynucleotide, host cells, expressing the construct and a pharmaceutical composition comprising the same.

The present invention relates to a method for the treatment of patients suffering from blood cancer, in particular from leukemia or lymphoma using the subcutaneous administration of a T cell engager comprising a CD19-binding domain.

### INCORPORATION BY REFERENCE OF THE SEQUENCE LISTING

This application contains, as a separate part of disclosure, a Sequence Listing in computer-readable form (Filename: 57331_SeqListing.xml; Size: 32,039 bytes; Created: Dec. 10, 2024) which is incorporated by reference herein in its entirety.

### BACKGROUND OF THE INVENTION

Cancer immunotherapies require a target antigen firmly bound to the surface of cancer cells in order to be active. By binding to the surface target, immunotherapeutics comprising cancer target-antigen specific binding domains can directly deliver a deadly signal to the cancer cell or indirectly by, for example, recruiting a cytotoxic T cell, if it is a T-cell engaging drug (T cell engager). In an ideal treatment scenario, a target antigen is abundantly present and accessible on every cancer cell and is absent, shielded or much less abundant on normal cells. Alternatively, a target antigen may be restricted to a certain lineage of normal cells and cancer cells derived therefrom, wherein the depletion of target antigen-positive normal cells is tolerable e.g., because of their recovery from target antigen-negative stem cells. These situations provide the basis for a therapeutic window in which a defined amount of the immunotherapeutic-based therapeutic effectively hits cancer cells but spares normal cells.

Though antibodies, which may form one of class of immunotherapeutics, and other therapeutics that comprise domains of antibodies or other commonly known derivatives of antibodies and fragments thereof, are effective means in treating many disorders, in particular cancer, their administration is not necessarily devoid of side effects. Adverse effects may cause a reversible or irreversible change in the health status of a patient. As adverse effects could potentially be harmful and lead to an interruption of a critically important therapy, it is highly desirable to avoid them. In clinical trials, a general distinction can be made between adverse effects (AEs) and serious adverse effects (SAEs). Specifically, adverse effects can be classified in 5 grades in accordance with the Common Terminology Criteria for Adverse Events (CTCAE) version 4. Grade 1 relates to mild AE, Grade 2 to moderate AE, Grade 3 to severe AE, Grade 4 to life-threatening or disabling AE, while Grade 5 means death related to AE. An adverse effect observed in antibody therapy is the occurrence of infusion-related side effects, such as the cytokine release syndrome (“CRS”). Other adverse side effects described to be associated with CRS are fatigue, vomiting, tachycardia, hypertension, back pain, but also central nervous system neurological reactions (CNS reactions), such as seizures, encephalopathy, cerebral edema, aseptic meningitis, and headache. Side effects that occur timely distinct side from CRS, often days later, are neurological side effects. While symptoms of CRS and neurological adverse events may resemble each other, their occurrence is quite different as known in the field.

Cytokine release and neurological reactions have not only been observed with monoclonal antibodies binding to the T cell receptor but also with a CD19×CD3 bispecific single chain T cell engager binding to the CD3 part of the T cell receptor (designated Blinatumomab (MT103) or AMG 103).

Blinatumomab is a B cell malignancy-directed, recombinant bispecific single-chain CD19×CD3 T cell engager that binds to CD19 on the surface of almost all B cells and B tumor cells and concomitantly can engage a T cell, thereby triggering the T-cell to kill the target B cell or B tumor cell. Blinatumomab consists of four immunoglobulin variable domains assembled into a single polypeptide chain. Two of the variable domains form the binding site for CD19, a cell surface antigen expressed on most B cells and B tumor cells. The other two variable domains form the binding site for the CD3 complex on T cells. Blinatumomab (trade name: Blincyto®) is designed to direct the body's cytotoxic, or cell-destroying, T cells against tumor cells, and is the first BiTE® (Bispecific T cell engager) molecule that received market approval.

As described for instance in WO 99/54440, adverse effects have been observed in a study performed with Blinatumomab applied in repeated bolus infusions to a patient with B-cell derived chronic lymphatic leukaemia (B-CLL). In order to try to better manage these undesired side effects, the mode of administration of the CD19×CD3 T cell engager has been changed in that it has been switched over from bolus infusion to a continuous intravenous administration of said antibody for a longer period of time.

According to authorization details of the European Medical Agency (EMA) (https://www.ema.europa.eu/en/medicines/human/EPAR/blincyto), Blincyto is given by infusion (drip) into a vein using a pump device. For treatment of relapsed or refractory B-precursor ALL, Blincyto is infused continuously during a treatment cycle of four weeks. Each cycle is separated by a two-week treatment-free interval. Patients who have no signs of cancer after two cycles may be treated with up to three additional cycles of Blincyto if the benefits outweigh the risks for the patient. For treatment of patients with minimal residual disease (MRD), the dose depends on the patient's bodyweight. Blincyto is infused continuously during a treatment cycle of four weeks. After receiving the first induction cycle patients may be treated for up to three additional treatment cycles, each one given after a two-week treatment-free interval.

Therefore, while immunotherapeutics, and in particular blinatumomab, have proven to be extremely efficient and safe, patients still require long periods of hospitalization. It would thus be very helpful to find a safe and efficient way to administer immunotherapeutics, particularly blinatumomab, in a way that is more convenient for patients, preferably a mode of administration that permits treatment periods to be made completely or partially outside of a hospital. The prevention of hospitalizing patients also reduces the need for the requirement of trained personnel during administration thereby lowering treatment administration costs. More preferably, such methods should also not require on-body devices such as pumping devices to be worn by the patient. This abrogates the risk of device-related infections and decreases the severity of infusion-related reactions. One mode of administration to address these issues would be the subcutaneous administration of blinatumomab. Subcutaneous administration helps preventing cIV infusion pump-related such as overdoses caused by incorrect pump settings and occlusion of intravenous lines. Further, while clinical trials have been initiated to administer blinatumomab subcutaneously (e.g., in clinical studies NCT02961881 and NCT04521231), safe and efficient dosing regimen to treat patients accordingly in terms of acceptable dosing regimen were so far unknown. Therefore, one of the main objectives underlying the present invention was the provision of a safe and efficient way of administering blinatumomab with tolerable side effects, for example no or only low and manageable CRS symptoms, by way of subcutaneous administration that does not require lengthy, i.e., costly, and psychologically challenging hospital-dependent treatments. Further, outpatient treatments with portable devices that are often difficult to handle can equally be avoided by the inventive methods, which can also be performed in community settings rather than in hospitals. This would lead to an overall improvement of patient convenience and their health-related quality of life. Still further, for an efficient and safe dosing regimen, it would also be beneficial to improve the pharmacokinetic profile of the drug, i.e., to extend the time-period in which a drug, such as blinatumomab, is present and can exert its pharmacological effects in the patient to be treated. Extending the pharmacokinetic profile means that interval between the administration of two individual doses of the drug can be extended. At the same time the activation and distribution profiles of the T cells should not rapidly peak and then decline but increase at a slower rate and reach plateau-like profiles. This would be preferable from a viewpoint of avoiding or reducing adverse events such as CRS and/or neurotoxic side effects. The above objectives have been achieved by the subject matter of the present invention.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. In each instance herein any of the terms “comprising”, “consisting essentially of” and “consisting of” may be replaced with either of the other two terms.

Several documents are cited throughout the text of this specification. Nothing herein is to be construed as an admission that the invention is not entitled to antedate the disclosure of the publications and patents cited throughout the text of this specification (including all patents, patent applications, scientific publications, manufacturer's specifications, instructions etc.). To the extent the cited material contradicts or is inconsistent with this specification, the specification will supersede any such material.

### DETAILED DESCRIPTION

In a 1^{st} embodiment, the present invention relates to a method of treating or ameliorating a lymphoma or a leukemia in a patient using a T cell engaging polypeptide construct binding to CD19 and comprising the steps:
Administering said T cell engaging polypeptide construct to said patient in a first treatment cycle;
Wherein at least two individual doses of a first quantity of said T cell engaging polypeptide construct are subcutaneously administered in a first predetermined period;
Optionally wherein at least two individual doses of a second quantity of said T cell engaging polypeptide construct are subcutaneously administered in a second predetermined period.

As used herein, the terms “first quantity”, “second quantity”, etc., define the individual amounts of a drug, e.g., blinatumomab, that is administered at a given point in time. In other words, combined “quantities” of the drug will be administered over a period of time, which, for the sake of the present disclosure, is defined as a “predetermined period” preceded by a specific number. This means that the present inventors have identified respective periods in which it is beneficial to administer a medication and thereby provide “predetermined period(s)” that are applied in steps of the methods and the uses of the present invention. Consequently, a “treatment cycle” is composed of various “predetermined periods”.

In a 2^{nd} embodiment, the present invention relates to a method according to any preceding embodiment, wherein said first treatment cycle is preceded by and/or followed by at least one additional treatment cycle. The additional treatment cycle may either precede the first treatment cycle or follow said cycle or both. In embodiments, the medication administered in the first treatment cycle is identical to the medication in the preceding and/or following cycles. The medication in these cycles may be administered using a different or the same administration mode, i.e. the drug may be administered subcutaneously as in the first treatment cycle, or it may be administered, for example, intravenously. In further embodiments, the medication comprises the administration of a B-cell depleting agent, particularly a T cell engager such as blinatumomab.

In a 3rd embodiment, the present invention relates to a method according to any of the preceding embodiments, wherein a third predetermined treatment-free period precedes and/or follows said first treatment cycle.

In a 4^{th} embodiment, the present invention relates to a method according to any of the preceding embodiments, wherein a fourth predetermined treatment-free period follows said first predetermined period.

In a 5^{th} embodiment, the present invention relates to a method according to any of the preceding embodiments, wherein the first quantity is administered in 2 to 9 doses.

In a 6^{th} embodiment, the present invention relates to a method according to any of the preceding embodiments, wherein the first predetermined period is 5 to 9 days, particularly 7 days.

In a 7^{th} embodiment, the present invention relates to a method according to any of the preceding embodiments, wherein the first quantity is either 10 to 80 μg, 20 to 80 μg, particularly 30 to 75 μg.

In an 8^{th} embodiment, the present invention relates to a method according to any of the preceding 1^{st} to 6^{th} embodiments, wherein the first quantity is either 100 μg to 800 μg, 200 to 700 μg, particularly about 112 μg, 225 μg, 450 μg, or 675 μg.

In a 9^{th} embodiment, the present invention relates to a method according to any of the preceding 1^{st} to 7^{th} embodiments, wherein the second predetermined period is 1 to 28 days, particularly 1 to 21 days.

In a 10^{th} embodiment, the present invention relates to a method according to any of the preceding 1^{st} to 7^{th} and 9^{th} embodiments, wherein the second quantity is 200 to 300 μg, particularly 225 to 275 μg, more particularly 250 μg.

In an 11^{th} embodiment, the present invention relates to a method according to any of the preceding 1^{st} to 7^{th}, and 9^{th} to 10^{th} embodiments, wherein the second quantity is administered 2 to 5 times weekly, particularly 3 times weekly.

In a 12^{th} embodiment, the present invention relates to a method according to any of the preceding 1^{st} to 7^{th}, and 9^{th} to 11^{th} embodiments, wherein the quantity administered in the at least one subsequent cycle is 200 to 300 μg, particularly 225 to 275 μg, more particularly 250 μg.

In a 13^{th} embodiment, the present invention relates to a method according to any of the preceding 1^{st} to 7^{th}, and 9^{th} to 12^{th} embodiments, wherein the quantity in the at least one subsequent cycle is administered 2 to 5 times weekly, preferably 3 times weekly, which optionally is administered subcutaneously.

In a 14^{th} embodiment, the present invention relates to a method according to any of the preceding embodiments, wherein the T cell engaging polypeptide construct binds to CD3.

In a 15^{th} embodiment, the present invention relates to a method according to any of the preceding embodiments, wherein the T cell engaging polypeptide construct binds to human and macaque CD3, particularly to the epsilon chain of the CD3 complex.

In a 16^{th} embodiment, the present invention relates to a method according to any of the preceding embodiments, wherein the T cell engaging polypeptide construct is a single chain polypeptide.

In a 17^{th} embodiment, the present invention relates to a method according to any of the preceding embodiments, wherein the T cell engaging polypeptide construct comprises the CDR regions depicted in SEQ ID NOs: 11 to 22.

In a 18^{th} embodiment, the present invention relates to a method according to any of the preceding embodiments, wherein the T cell engaging polypeptide construct comprises the VH and VL regions depicted in SEQ ID NOs: 3, 5, 7, and 9.

In a 19^{th} embodiment, the present invention relates to a method according to any of the preceding embodiments, wherein the lymphoma or leukemia is selected from the group comprising Non-Hodgkin Lymphoma and Acute Lymphoblastic Leukemia.

In a 20^{th} embodiment, the present invention relates to a method according to any of the preceding embodiments, wherein the patient has been subject to at least one of the following treatments preceding the subcutaneous administration scheme set forth in any of the preceding embodiments, wherein said preceding treatments is selected from the group comprising cIV administration of a B-cell depleting agent, particularly of blinatumomab, administration with a CD19-specific CAR T-cell therapy, and/or administration of a CD20-targeting agent, optionally preceded or in combination with a chemotherapy.

In a 21^{st} embodiment, the present invention relates to a method according to any of the preceding 1^{st} to 7^{th}, 9^{th} to 20^{th} embodiments, wherein the first quantity is 30 to 50 μg, particularly 40 μg and the patient suffers from (relapsed/refractory) B-cell precursor acute lymphoblastic leukemia.

In a 22^{nd} embodiment, the present invention relates to a method according to any of the preceding 1^{st} to 6^{th}, and 8^{th}, 14^{th} to 20^{th} embodiments, wherein patient suffers from (relapsed/refractory, R/R) indolent Non-Hodgkin's Lymphoma.

In a specific embodiment, the present invention relates to a method of treating an adult patient suffering from Relapsed or Refractory B Cell Precursor Acute Lymphoblastic Leukemia (R/R B-ALL) with blinatumomab comprising the steps:
Selecting a patient suffering from R/R B-ALL;
Administering blinatumomab subcutaneously to said patient in a first treatment cycle that is 34 days long and includes a 26-day treatment period and an 8-day treatment-free interval,
Wherein said patient receives 40 μg of SC blinatumomab once daily on days 1-7 and then 250 μg 3 times weekly (MWF) on days 8-26;
Followed by at least one additional cycle, wherein said patient receives 250 μg 3 times weekly during the entire treatment period.

In another specific embodiment, the present invention relates to a method of treating an adult patient suffering from Relapsed or Refractory B Cell Precursor Acute Lymphoblastic Leukemia (R/R B-ALL) with blinatumomab comprising the steps:
Selecting a patient suffering from R/R B-ALL;
Administering blinatumomab subcutaneously to said patient in a first treatment cycle that is 34 days long and includes a 26-day treatment period and an 8-day treatment-free interval,
Wherein said patient receives 120 μg of SC blinatumomab once daily on days 1-7 and then 250 μg 3 times weekly (MWF) on days 8-26;
Optionally followed by at least one additional cycle, wherein said patient receives 250 μg 3 times weekly during the entire treatment period.

In another specific embodiment, the present invention relates to a method of treating an adult patient suffering from Relapsed or Refractory B Cell Precursor Acute Lymphoblastic Leukemia (R/R B-ALL) with blinatumomab comprising the steps:
Selecting a patient suffering from R/R B-ALL;
Administering blinatumomab subcutaneously to said patient in a first treatment cycle that is 34 days long and includes a 26-day treatment period and an 8-day treatment-free interval,
Wherein said patient receives 250 μg of SC blinatumomab once daily on days 1-7 and then 500 μg 3 times weekly (MWF) on days 8-26;
Optionally followed by at least one additional cycle, wherein said patient receives 500 μg 3 times weekly during the entire treatment period.

In another specific embodiment, the present invention relates to a method of treating an adult patient suffering from Relapsed or Refractory B Cell Precursor Acute Lymphoblastic Leukemia (R/R B-ALL) with blinatumomab comprising the steps:
Selecting a patient suffering from R/R B-ALL;
Administering blinatumomab subcutaneously to said patient in a first treatment cycle that is 34 days long and includes a 26-day treatment period and an 8-day treatment-free interval,
Wherein said patient receives 500 μg of SC blinatumomab once daily on days 1-7 and then 1000 μg 3 times weekly (MWF) on days 8-26;
Optionally followed by at least one additional cycle, wherein said patient receives 1000 μg 3 times weekly during the entire treatment period.

In another specific embodiment, the present invention relates to a method of treating an adult patient suffering from relapsed or refractory indolent Non-Hodgkin's Lymphoma (NHL) with blinatumomab comprising the steps:
Selecting a patient suffering from relapsed or refractory indolent NHL;
Administering blinatumomab subcutaneously to said patient in a first treatment cycle that is 5 to 6 weeks long for about 6 days,
Wherein said patient receives about 675 μg blinatumomab subcutaneously in three doses on days 1, 3 and 5;
Preceded by a cIV step-dosing run-in period of three weeks, wherein the patient is administered 9 μg/d during the first week, followed by 28 μg/d during the second week, and 112 μg/d in the third week;
Followed by a period of 2 days without administration of blinatumomab; and
112 μg/d in the fifth and sixth week;
Optionally followed by a 2 weeks blinatumomab treatment-free period, and
Further optionally followed by an additional 3 to 6 weeks long treatment cycle with blinatumomab, wherein the patient receives 9 μg/d during the first week, followed by 28 μg/d during the second week, and 112 μg/d in the third week.

In further embodiments, the present invention relates to uses of the herein described B cell-depleting agents, particularly blinatumumab, in the preparation of a medicament wherein said medicament is suitably adapted or prepared for administration to a patient in need thereof according to any of the above-mentioned methods/dosing regimen.

In still further embodiments, the present invention relates to apparatus that either comprising a containment device or that can be connected with such a containment device, wherein the containment device (e.g., a syringe, a vessel, etc.) comprises an of the herein described B cell-depleting agents, particularly blinatumumab, suitably adapted or prepared for administration to a patient in need thereof according to any of the above-mentioned methods/dosing regimen.

Yet other embodiments of the invention relate to kits-of-parts that comprise dosage units suitably adapted or prepared for administration to a patient in need thereof according to any of the above-mentioned methods/dosing regimen. Such kits may comprise an injection device, e.g., a syringe, injection needles, vessels comprising liquids for reconstitution of freeze-dried active agents and/or additives, particularly for blinatumumab, or ready-prepared formulations in individual dosage units. Individual dosage units may be color-coded to select the appropriate dose to be administered at a given time-point of the inventive dosing regimen. Of course, kits may also comprise technical information in form of hard copies or in digitalized form.

Further embodiments are listed in the appended claims.

### Non-Hodgkin's Lymphoma (NHL)

Within the meaning of the invention, the term “B cell non-Hodgkin lymphoma” or “B cell derived non-Hodgkin lymphoma” comprises both indolent and aggressive B cell non-Hodgkin lymphoma (B NHL). The term “indolent or aggressive B cell non-Hodgkin lymphoma (B NHL)” as used herein represents malignant B cell-derived tumorous diseases. Indolent B NHL are low malignant lymphomas. Aggressive B-NHL are high malignant lymphomas. The B cell non-Hodgkin lymphoma (B NHL) may be a follicular lymphoma, lymphoplasmacytic lymphoma, marginal zone cell lymphoma, mantle cell lymphoma (MCL), diffuse large B cell lymphoma (DLBCL), Burkitt's lymphoma, small lymphocytic lymphoma (SLL/CLL) and any other B cell derived subtype. The term “B cell leukemia” as used herein may advantageously be any B cell leukaemia (e.g., chronic lymphocytic leukaemia or acute lymphocytic leukaemia). For further reference see e.g., http://www.cancer.org. Preferably, indolent non-Hodgkin B cell lymphoma may be treated with a T cell engaging polypeptide construct directed against both human CD3 and human CD19 as demonstrated in the examples below.

### Acute Lymphoblastic Leukemia (ALL)

“Acute lymphoblastic leukemia” or “ALL”, also known as acute lymphocytic leukemia or acute lymphoid leukemia, generally refers to an acute form of leukemia which is typically characterized by the overproduction and/or accumulation of cancerous, immature white blood cells (also referred to as lymphoblasts). As used herein, the term “ALL” includes acute, refractory and relapsed ALL. The term “refractory ALL” as used herein means resistance of the ALL to conventional or standard ALL therapy, such as chemotherapy and/or hematopoietic stem cell transplantation (HSCT), i.e., the conventional or standard ALL therapy is not able to ultimately cure all ALL patients. The term “relapsed ALL” as used herein denotes the return of signs and symptoms of the ALL disease after a patient has enjoyed a remission. For example, after conventional ALL treatment using chemotherapy and/or HSCT, an ALL patient may go into remission with no sign or symptom of the ALL, remains in remission for a couple of years, but then suffers a relapse and has to be treated once again for ALL. The term “ALL” as used herein also includes minimal residual disease (MRD) in a patient with ALL, i.e., the presence of a small numbers of cancerous lymphoblasts remaining in the patient during treatment, or after treatment when the patient is in remission.

The term “ALL” generally encompasses B-cell ALL and T-cell ALL. The term “cancerous” is used herein interchangeably with the term “malignant” to designate cells that are not self-limited in their growth, are capable of invading into adjacent tissues, and may be capable of spreading to distant tissues (metastasizing).

It is envisaged that the methods and uses disclosed herein are particularly useful for treating B-cell ALL, including B-precursor ALL, such as pro-B ALL, pre-B ALL, or common ALL (CALL), and mature B-cell ALL (Burkitt leukemia). The term “ALL” includes both pediatric ALL and adult ALL. The means and methods of the present invention are in particular envisaged to be useful for treatment of relapsed and/or refractory adult B-precursor ALL.

The term “patient” includes all mammals, but is not limited to mouse, rat, dog, horse, camel, primates, etc., primates being preferred and humans, including children and adults, being most preferred. When used herein, the term “subject” is used interchangeably with the term “patient”. What is disclosed with reference to a “patient” herein also applies to a group of patients, mutatis mutandis.

The term “pediatric ALL” or “pediatric ALL patient” as referred to herein denotes children aged from one month to 18 years. The indicated age is to be understood as the age of the children at diagnosis of the ALL disease. Both time intervals specifically include the upper limit and also the lower limit. This means that for example a time interval “from one month to 18 years” includes “one month” and “18 years”. WO 2010/052013 provides means and methods for treating pediatric or childhood ALL, particularly refractory and/or relapsed pediatric ALL.

The term “adult ALL” or “adult ALL patient” as referred to herein denotes adults aged more than 18 years, i.e. patients aged 19, 20, 21, 22, 23, 24, 25, 30, 35, 40, or 50 years or more. Even patients with 70, 75, 80, 85, 90, 100 years or older may be treated by the methods and means of the invention. The indicated age is to be understood as the age of the adult at diagnosis of the ALL disease. WO 2010/052014 provides means and methods for treating adult ALL.

### T Cell Engaging Polypeptide Construct

The uses and methods of the present invention involve administration of a (therapeutically effective amount of) T cell engaging polypeptide construct to a patient (or a group of patients). As used herein, a T cell engaging polypeptide construct may comprise at least one domain that binds to a target on a T cell surface, e.g., a part of the CD3 molecule. Another domain of the T cell engaging polypeptide construct may comprise at least one domain that binds to a structure on a cell that should be attacked by a T cell. An exemplary structure on the surface of such a cell, e.g. a cancer cell, may comprise a protein such as a tumor-associated antigen. It is important for the mechanism of action of the T cell engaging polypeptide construct that it brings a T cell into proximity with another cell that expresses said structure on the surface of such a cell, e.g. a cancer cell, that expresses a tumor-associated antigen, that the T cell exerts cytolytic actions (e.g. secretes perforin or other substances produced by cytotoxic T cells, which are known in the art). An example of a T cell engaging polypeptide construct is a bispecific T cell engager also known in the art as BiTE® molecule (which is a registered trademark of Amgen Inc.). On the other hand, the T cell engaging polypeptide construct may also be a protein that is expressed by the cytolytic T cell. The protein may be a non-naturally expressed T-cell receptor that is expressed as result of genetic engineering, which binds, e.g. to a tumor-associated antigen. The latter T cell engaging polypeptide construct is commonly used in the context of CAR-T cell therapy. Further, as used herein the term “T cell engaging polypeptide construct” means that it refers to a construct that is not normally found in nature, i.e. it was designed and produced using technological means. It is also possible that the polypeptide construct comprises non-peptidic elements, e.g. elements that are not comprised of amino acids, for example organic molecules that are toxins, linker molecules that consist of molecules that are not amino acids, or half-life extending moieties such as biotin.

It is noted that, instead of referring to a T cell engaging polypeptide construct, it is also contemplated in the herein described uses and methods to administer NK cell engaging polypeptides, which activate NK cells to exert their cytolytic potential upon formation of a close enough contact between a target cell and an NK cell. In such a case, the polypeptide binds to a target antigen such as CD19 and to an NK cell-specific antigen establishing a proximity between target cell and effector cell that permits an activated NK cell to effectively lyse or otherwise kill the target cell.

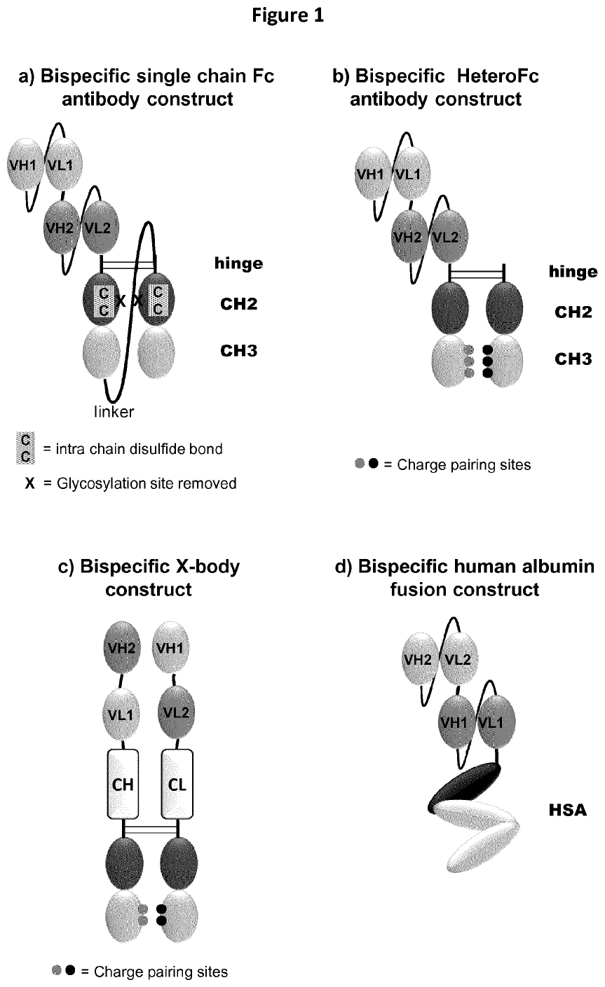

The terms “bispecific T cell engager” or “single chain bispecific T cell engager” or related terms in accordance with the present invention mean T cell engager constructs resulting from joining at least two antibody variable regions in a single polypeptide chain devoid of the constant and/or Fc portion(s) present in full immunoglobulins. A “linker” as used herein connects V domains of the same specificity, whereas a “spacer” as used herein connects V domains of different specificities. For example, a bispecific single chain T cell engager may be a construct with a total of two antibody variable regions, for example two VH regions, each capable of specifically binding to a separate antigen, and connected with one another through a short (usually less than 10 amino acids) synthetic polypeptide spacer such that the two antibody variable regions with their interposed spacer exist as a single contiguous polypeptide chain. Another example of a bispecific single chain T cell engager may be a single polypeptide chain with three antibody variable regions. Here, two antibody variable regions, for example one VH and one VL, may make up an scFv, wherein the two antibody variable regions are connected to one another via a synthetic polypeptide linker, the latter often being genetically engineered to be minimally immunogenic while remaining maximally resistant to proteolysis. This scFv is capable of specifically binding to a particular antigen, and is connected to a further antibody variable region, for example a VH region, capable of binding to a different antigen than that bound by the scFv. Yet another example of a bispecific single chain T cell engager may be a single polypeptide chain with four antibody variable regions. Here, the first two antibody variable regions, for example a VH region and a VL region, may form one scFv capable of binding to one antigTeen, whereas the second VH region and VL region may form a second scFv capable of binding to another antigen. Within a single contiguous polypeptide chain, individual antibody variable regions of one specificity may advantageously be separated by a synthetic polypeptide linker as described above, whereas the respective scFvs may advantageously be separated by a short polypeptide spacer as described above. Non-limiting examples of bispecific single chain T cell engagers as well as methods for producing them are shown in WO 99/54440, WO 2004/106381, Mack, J. Immunol. (1997), 158, 3965-70; Mack, PNAS, (1995), 92, 7021-5; Kufer, Cancer Immunol. Immunother., (1997), 45, 193-7; Löffler, Blood, (2000), 95, 6, 2098-103; Brühl, J. Immunol., (2001), 166, 2420-2426. As used herein, the above terms can also be extended to T cell engaging molecules that have more than one domain that binds to a tumor-associated antigen. This means that at least two domains can bind to at least two antigenic sides on the same or different tumor associated targets. Similarly, the T cell engaging part of the respective molecule may bind to at least two antigens expressed by a T cell (or, as the case may be, an NK cell). These two antigens may be identical, e.g. two identical antigens on two identical cell surface proteins or two different antigens on either the same protein or on different proteins may be bound. It is noted that T cell engagers according to the present invention may comprise a half-life extending domain, such as disclosed, for example, in WO2017/134140, particularly those disclosed in the claims and in FIG. 1, which explicitly refers to certain Fc constructs, and T cell engagers comprising a human serum albumin component.

As used herein, “human CD3” denotes an antigen that is expressed on human T cells as part of the multimolecular T cell receptor complex, the CD3 consisting of five different chains: CD3-epsilon, CD3-gamma, CD3-delta, CD3-eta and CD3 zeta. Clustering of CD3 on T cells e.g. by anti-CD3 antibodies leads to T cell activation similar to the binding of an antigen but independent from the clonal specificity of the T cell subset, as described above. Thus, the term “a bispecific single chain T cell engager polypeptide construct specifically binding with one of its specificities the human CD3 antigen” as used herein relates to a CD3-specific construct capable of binding to the human CD3 complex expressed on human T cells and capable of inducing elimination/lysis of target cells, wherein such target cells carry/display an antigen which is bound by the other, non-CD3-binding portion of the bispecific single chain T cell engager. Binding of the CD3 complex by CD3-specific binders (e.g. a bispecific single chain T cell engager polypeptide construct as administered according to the pharmaceutical means and methods of the invention) leads to activation of T cells as known in the art; see e.g. WO 99/54440 or WO 2004/106381. Accordingly, a construct appropriate for the pharmaceutical means and methods of the invention is advantageously able to eliminate/lyse target cells in vivo and/or in vitro. Corresponding target cells comprise cells expressing a tumor antigen, such as CD19, which is recognized by the second specificity (i.e. the non-CD3-binding portion of the bispecific single chain T cell engager) of the mentioned construct. Preferably, said second specificity is for human CD19 which has already been described in WO 99/54440 or WO 2004/106381. According to this embodiment, each antigen-specific portion of the bispecific single chain T cell engager comprises an antibody VH region and an antibody VL region. Advantageous variants of this bispecific single chain T cell engager are from N terminus to C terminus:
VL(CD19)-VH(CD19)-VH(CD3)-VL(CD3),
VH(CD19)-VL(CD19)-VH(CD3)-VL(CD3),
VH(CD3)-VL(CD3)-VH(CD19)-VL(CD19), or
VH(CD3)-VL(CD3)-VL(CD19)-VH(CD19).

More particularly, within the meaning of the invention, the term “specifically binding” or related terms such as “specificity” is/are to be understood as being characterized primarily by two parameters: a qualitative parameter (the binding epitope, or where an antibody or an inventive T cell engager binds) and a quantitative parameter (the binding affinity, or how strongly this antibody binds where it does). Which epitope is bound by an antibody can advantageously be determined by e.g. FACS methodology, ELISA, peptide-spot epitope mapping, or mass spectroscopy. The strength of antibody or an inventive T cell engager binding to a particular epitope may advantageously be determined by e.g. known Biacore and/or ELISA methodologies. A combination of such techniques allows the calculation of a signal:noise ratio as a representative measure of binding specificity. In such a signal:noise ratio, the signal represents the strength of antibody or T cell engager binding to the epitope of interest, whereas the noise represents the strength of antibody or T cell engager binding to other, non-related epitopes differing from the epitope of interest. A signal:noise ratio of, for example at least 50, but preferably about 80 for a respective epitope of interest as determined e.g. by Biacore, ELISA or FACS may be taken as an indication that the antibody evaluated binds the epitope of interest in a specific manner, i.e. is a “specific binder”. The term “binding to/interacting with” may also relate to a conformational epitope, a structural epitope or a discountinuous epitope consisting of two regions of the human target molecules or parts thereof. In context of this invention, a conformational epitope is defined by two or more discrete amino acid sequences separated in the primary sequence which come together on the surface of the molecule when the polypeptide folds to the native protein (Sela, (1969) Science 166, 1365 and Laver, (1990) Cell 61, 553-6). The term “discontinuous epitope” means in context of the invention non-linear epitopes that are assembled from residues from distant portions of the polypeptide chain. These residues come together on the surface of the molecule when the polypeptide chain folds into a three-dimensional structure to constitute a conformational/structural epitope.

According to the present invention the term “variable region” used in the context with Ig-derived antigen-interaction comprises fragments and derivatives of polypeptides which at least comprise one CDR derived from an antibody, antibody fragment or derivative thereof. It is envisaged by the invention, that said at least one CDR is preferably a CDR3, more preferably the CDR3 of the heavy chain of an antibody (CDR-H3). However, other antibody derived CDRs are also particularly comprised by the term “variable region”.

### B-Cell Depleting Agent/T Cell Engaging Polypeptide Constructs

The uses and methods of the present invention involve administration of a (therapeutically effective amount of) B cell depleting agent to a patient (or a group of patients). In various embodiments, the uses and methods of the present invention relate to T cell engaging polypeptide constructs, which are the B-cell depleting agents principally used in the context of the present invention.

In general, any route of administration is conceivable depending, e.g., on the formulation, bioavailability and mechanism of action of the B-cell depleting agent. However, in the context of the present invention, the B-cell depleting agent can be transdermally, and preferably, subcutaneously. Thus, in the context of the uses and methods of present invention, subcutaneous administration is used within the context of the claimed uses and methods unless reference is made to steps in the method that either precede or follow a cycle of subcutaneously administering the T cell engager polypeptide construct. By “therapeutically effective amount” is meant an amount of the B-cell depleting agent that elicits a desired therapeutic effect, e.g., alleviation or amelioration (complete or partial) of the symptoms or condition of the patient (or group of patients), or any other desired improvement in the patient's (or group of patients') symptoms, disease or condition. The exact amount dose may depend on, e.g., age, body weight, general health, sex, diet, drug interaction and the severity of the condition, as will be ascertainable with routine experimentation by those skilled in the art.

The term “B cell depleting agent” in general refers to an agent capable of reducing and/or controlling the number of B-cells in a patient, and particularly to T cell engaging polypeptide constructs

The term thus includes agents that directly or indirectly destroy of some or all B-cells, e.g. by induction of cell death signals, antibody dependent cell-mediated cytotoxicity (ADCC), complement dependent cytotoxicity (CDC), or engagement of cytotoxic T-cells, and agents that block B cell activation or development. The term “B-cell” includes progenitor (or pre-pro) B cells, early pro (or pre-pre)-B cells, late pro (or pre-pre)-B cells, large pre-B cells, small pre-B cells, immature B cells and mature B cells. B cell depletion can be partial or complete, i.e. affect all B-cells or subpopulations of B-cells. Preferred B-cell depleting agents for use in the methods of the invention can reduce (or maintain) the level of B-cells in the blood of a patient (or group of patients) within a predefined period of time to one B-cell/ml serum or less as ascertainable by the skilled person using routine experimentation as described herein. It is in particular envisaged that B-cell depleting agents used in the methods of the invention are capable of depleting peripheral CD19+ B-cells.

B-cell depleting agents are known in the art and include, without limitation, co-stimulation blockers (abatacept and 7-related protein-1), cytokines (tocilizumab and baminercept), B cell receptor-targeted agents (abetimus and edratide), agents targeting CD20, CD22, CD19, CD40-CD40L, B cell activating factor belonging to the TNF family (BAFF) or A proliferation-inducing ligand (APRIL). In accordance with the foregoing, exemplary B-cell depleting agents useful in the methods of the invention include anti-CD20 agents (e.g., anti-CD20 antibodies such as rituximab, ofatumumab, ocrelizumab, veltuzumab, tositumomab, ibritumomab), anti-CD25 agents (e.g., anti-CD25 antibodies such as alemtuzumab), BAFF inhibitors (e.g., belimumab, atacicept), anti-CD154 agents (e.g. anti-CD154 antibodies such as ruplizumab, toralizumab), anti-CD19 agents (e.g., MDX-1342), anti-CD22 agents (e.g., epratuzumab) and anti-thymocyte globulin (ATG).

In particular, agents targeting CD19 and CD22 are envisaged, such as bispecific CD19×CD22 single chain polypeptides, and particularly blinatumomab. Without wishing to be bound by theory, it is thought that blinatumomab transiently links CD19⁺ B cells to CD3⁺ T-cells, thereby inducing T-cell mediated serial lysis of B-cells and concomitant T-cell proliferation.

Blinatumomab comprises the
(a) anti-CD3 CDRs of the heavy chain shown as CD3 CDR-H1 in SEQ ID NO: 11 (GYTFTRYTMH), CD3 CDR-H2 in SEQ ID NO: 12 (YINPSRGYTNYNQKFKD) and CD3 CDR-H3 in SEQ ID NO: 13 (YYDDHYCLDY); and/or
(b) anti-CD3 CDRs of the light chain shown as CD3 CDR-L1 in SEQ ID NO: 14 (RASSSVSYMN), CD3 CDR-L2 in SEQ ID NO: 15 (DTSKVAS) and CD3 CDR-L3 in SEQ ID NO: 16 (QQWSSNPLT); and/or
(c) anti-CD19 CDRs of the heavy chain shown as CD19 CDR-H1 in SEQ ID NO: 17 (GYAFSSYWMN), CD19 CDR-H2 in SEQ ID NO: 18 (QIWPGDGDTNYNGKFKG) and CD19 CDR-H3 in SEQ ID NO: 19 (RETTTVGRYYYAMDY); and/or
(d) anti-CD19 CDRs of the light chain shown as CD19 CDR-L1 in SEQ ID NO: 20 (KASQSVDYDGDSYLN), CD19 CDR-L2 in SEQ ID NO: 21 (DASNLVS) and CD19 CDR-L3 in SEQ ID NO: 22 (QQSTEDPWT).

In the alternative, Blinatumomab comprises the
(a) CD19 binding variable heavy chain shown in SEQ ID NO: 3 (nucleotide sequence is shown in SEQ ID NO: 4); and/or
(b) CD19 binding variable light chain shown in SEQ ID NO: 5 (nucleotide sequence is shown in SEQ ID NO: 6); and/or
(c) CD3 binding variable heavy chain shown in SEQ ID NO: 7 (nucleotide sequence is shown in SEQ ID NO: 8); and/or
(d) CD3 binding variable light chain shown in SEQ ID NO: 9 (nucleotide sequence is shown in SEQ ID NO: 10).

In a further alternative, Blinatumomab comprises an amino acid sequence selected from the group consisting of
(a) an amino acid sequence as depicted in SEQ ID NO: 1;
(b) an amino acid sequence encoded by a nucleic acid sequence as shown in SEQ ID NO: 2;
(c) an amino acid sequence encoded by a nucleic acid sequence having at least 70%, 80%, 90%, 95% or 99% identity to a nucleic acid sequence of (b), wherein said amino acid sequence is capable of binding to CD3 and CD19; and
(d) an amino acid sequence encoded by a nucleic acid sequence which is degenerate as a result of the genetic code to a nucleotide sequence of (b), wherein said amino acid sequence is capable of binding to CD3 and CD19.

An alternative to Blinatumomab as B-cell depleting agent is a CD3×CD19 antibody, wherein the CD3 binding molecule thereof preferably comprises a VL region comprising CDR-L1, CDR-L2 and CDR-L3 selected from:
(a) CDR-L1 as depicted in SEQ ID NO: 27 of WO 2008/119567, CDR-L2 as depicted in SEQ ID NO: 28 of WO 2008/119567 and CDR-L3 as depicted in SEQ ID NO: 29 of WO 2008/119567;
(b) CDR-L1 as depicted in SEQ ID NO: 117 of WO 2008/119567, CDR-L2 as depicted in SEQ ID NO: 118 of WO 2008/119567 and CDR-L3 as depicted in SEQ ID NO: 119 of WO 2008/119567; and
(c) CDR-L1 as depicted in SEQ ID NO: 153 of WO 2008/119567, CDR-L2 as depicted in SEQ ID NO: 154 of WO 2008/119567 and CDR-L3 as depicted in SEQ ID NO: 155 of WO 2008/119567; and/or comprises a VH region comprising CDR-H 1, CDR-H2 and CDR-H3 selected from:
(a) CDR-H1 as depicted in SEQ ID NO: 12 of WO 2008/119567, CDR-H2 as depicted in SEQ ID NO: 13 of WO 2008/119567 and CDR-H3 as depicted in SEQ ID NO: 14 of WO 2008/119567;
(b) CDR-H1 as depicted in SEQ ID NO: 30 of WO 2008/119567, CDR-H2 as depicted in SEQ ID NO: 31 of WO 2008/119567 and CDR-H3 as depicted in SEQ ID NO: 32 of WO 2008/119567;
(c) CDR-H1 as depicted in SEQ ID NO: 48 of WO 2008/119567, CDR-H2 as depicted in SEQ ID NO: 49 of WO 2008/119567 and CDR-H3 as depicted in SEQ ID NO: 50 of WO 2008/119567;
(d) CDR-H1 as depicted in SEQ ID NO: 66 of WO 2008/119567, CDR-H2 as depicted in SEQ ID NO: 67 of WO 2008/119567 and CDR-H3 as depicted in SEQ ID NO: 68 of WO 2008/119567;
(e) CDR-H1 as depicted in SEQ ID NO: 84 of WO 2008/119567, CDR-H2 as depicted in SEQ ID NO: 85 of WO 2008/119567 and CDR-H3 as depicted in SEQ ID NO: 86 of WO 2008/119567;
(f) CDR-H1 as depicted in SEQ ID NO: 102 of WO 2008/119567, CDR-H2 as depicted in SEQ ID NO: 103 of WO 2008/119567 and CDR-H3 as depicted in SEQ ID NO: 104 of WO 2008/119567;
(g) CDR-H1 as depicted in SEQ ID NO: 120 of WO 2008/119567, CDR-H2 as depicted in SEQ ID NO: 121 of WO 2008/119567 and CDR-H3 as depicted in SEQ ID NO: 122 of WO 2008/119567;
(h) CDR-H1 as depicted in SEQ ID NO: 138 of WO 2008/119567, CDR-H2 as depicted in SEQ ID NO: 139 of WO 2008/119567 and CDR-H3 as depicted in SEQ ID NO: 140 of WO 2008/119567;
(i) CDR-H1 as depicted in SEQ ID NO: 156 of WO 2008/119567, CDR-H2 as depicted in SEQ ID NO: 157 of WO 2008/119567 and CDR-H3 as depicted in SEQ ID NO: 158 of WO 2008/119567; and
(j) CDR-H1 as depicted in SEQ ID NO: 174 of WO 2008/119567, CDR-H2 as depicted in SEQ ID NO: 175 of WO 2008/119567 and CDR-H3 as depicted in SEQ ID NO: 176 of WO 2008/119567.

The CD3 binding molecule of a CD3×CD19 antibody may preferably comprise a VL region selected from the group consisting of a VL region as depicted in SEQ ID NO: 35, 39, 125, 129, 161 or 165 of WO 2008/119567; and/or comprise a VH region selected from the group consisting of a VH region as depicted in SEQ ID NO: 15, 19, 33, 37, 51, 55, 69, 73, 87, 91, 105, 109, 123, 127, 141, 145, 159, 163, 177 or 181 of WO 2008/119567.

The CD3 binding molecule of a CD3×CD19 antibody may preferably comprise a VL region and a VH region selected from the group consisting of:
(a) a VL region as depicted in SEQ ID NO: 17 or 21 of WO 2008/119567 and a VH region as depicted in SEQ ID NO: 15 or 19 of WO 2008/119567;
(b) a VL region as depicted in SEQ ID NO: 35 or 39 of WO 2008/119567 and a VH region as depicted in SEQ ID NO: 33 or 37 of WO 2008/119567;
(c) a VL region as depicted in SEQ ID NO: 53 or 57 of WO 2008/119567 and a VH region as depicted in SEQ ID NO: 51 or 55 of WO 2008/119567;
(d) a VL region as depicted in SEQ ID NO: 71 or 75 of WO 2008/119567 and a VH region as depicted in SEQ ID NO: 69 or 73 of WO 2008/119567;
(e) a VL region as depicted in SEQ ID NO: 89 or 93 of WO 2008/119567 and a VH region as depicted in SEQ ID NO: 87 or 91 of WO 2008/119567;
(f) a VL region as depicted in SEQ ID NO: 107 or 111 of WO 2008/119567 and a VH region as depicted in SEQ ID NO: 105 or 109 of WO 2008/119567;
(g) a VL region as depicted in SEQ ID NO: 125 or 129 of WO 2008/119567 and a VH region as depicted in SEQ ID NO: 123 or 127 of WO 2008/119567;
(h) a VL region as depicted in SEQ ID NO: 143 or 147 of WO 2008/119567 and a VH region as depicted in SEQ ID NO: 141 or 145 of WO 2008/119567;
(i) a VL region as depicted in SEQ ID NO: 161 or 165 of WO 2008/119567 and a VH region as depicted in SEQ ID NO: 159 or 163 of WO 2008/119567; and
(j) a VL region as depicted in SEQ ID NO: 179 or 183 of WO 2008/119567 and a VH region as depicted in SEQ ID NO: 177 or 181 of WO 2008/119567.

The CD3 binding molecule of a CD3×CD19 antibody may preferably comprise an amino acid sequence selected from the group consisting of SEQ ID NOs: 23, 25, 41, 43, 59, 61, 77, 79, 95, 97, 113, 115, 131, 133, 149, 151, 167, 169, 185 or 187 of WO 2008/119567.

The CD19 binding molecule of a CD19×CD3 antibody is preferably characterized by the VH and/or VL regions or CDRs as described herein for Blinatumomab.

Whether a B-cell depleting agent is suitable for use in the inventive methods is readily ascertainable by the skilled person in the art using routine experimentation. E.g., the choice of a suitable B-cell depleting agent can depend on the type of leukemia or lymphoma to be treated, and in particular the expression profile of the expanded B-cell population(s). For example, if the the leukemia is ALL and is characterized by an expanding CD19⁺ B cell population, use of a B-cell depleting agent targeting CD19 is likely to be useful.

It is envisaged that the number of B-cells in the blood of the patient (or group of patients) remains or falls below one B cell/ml serum within the predefined period of time as defined herein.

In general, B-cell numbers can be evaluated through several techniques available in the art, e.g. using the white blood cell (WBC, or leukocytes) count and differential. White blood cells can be counted manually in hemocytometes (Neubauer chamber) or with automated counters. To determine the differential, a drop of blood can be thinly spread over a glass slide, air dried, and stained with a Romanofsky stain, most commonly the Wright or May-Grunewald-Giemsa technique. Cells are then counted and classified using morphologic examination and/or histochemistry as described in Blumenreich M S. The White Blood Cell and Differential Count. In: Walker H K, Hall W D, Hurst J W, editors. Clinical Methods: The History, Physical, and Laboratory Examinations. 3rd edition. Boston: Butterworths; 1990. Chapter 153. Alternatively, leukocytes are isolated from a blood sample and stained with fluorescent-labeled antibodies against lymphocyte cell surface markers and subsequently analyzed by flow cytometry as described in the appended examples. Percentages of each type of lymphocyte are multiplied with absolute lymphocyte numbers to calculate absolute cell numbers for each lymphocyte subpopulation.

It is envisaged that the number of B-cells in the blood of the treated patient (or treated group of patients) remains or falls below one B-cell/ml serum within a predefined period of time after the initial treatment with said B-cell depleting agent. The initial treatment with the applied B-cell depleting agent preferably means the first treatment with said applied B-cell depleting agent, i.e. the patient (or group of patients) has not received the applied B-cell depleting agent before. Said patient (or group of patients) may, however, have received further ALL treatments as described elsewhere herein before. It is also conceivable that the patient (or group of patients) has received another B-cell depleting agent before the initial treatment with the applied B-cell depleting agent. E.g., when a first B-cell depleting agent has no therapeutic effect, and/or fails to reduce (or maintain) the number of B-cells in the blood of a patient (or group of patients) to one B-cell/ml serum or less, a second B-cell depleting agent may be used. The initial treatment with the second B-cell depleting agent will then start on the first day of treating the patient (or group of patients) with the second B-cell depleting agent. That is, it is conceivable to apply the methods of the invention repeatedly (i.e., in several cycles) with different B-cell depleting agents, the “initial treatment” starting at the day of first treatment with the B-cell depleting agent of the respective cycle. The term “different” B-cell depleting agent also includes the same B-cell depleting agent as used in a preceding cycle, but in a different formulation, concentrations, or the like. It is also conceivable to repeat several cycles of treatment with the same B-cell depleting agent until the desired level of B-cells in the blood of the patient (or group of patients) is achieved.

The “predefined period of time” in which the number of B-cells remains or falls below one B-cell/ml serum is envisaged to be 15 days or less, i.e. 14, 13, 12, 11, 10, 9, 8, 7, 6, 5, 4, 3 days or less. The length of the predefined period of time is ascertainable by the skilled person in the art and may depend on the applied B-cell depleting agent, its concentration, treatment regimen, the type of leukemia or lymphoma, e.g. ALL, to be treated, and the like. Without wishing to be bound by theory, it is thought that when the number of B-cells can be adjusted to the desired number of one B-cell/ml serum or less within the predefined period of time, subsequent treatment is likely to be effective. A possible reason is the expansion of the T- and/or TEM-cell population in patients exhibiting a clearance of (peripheral) B cells, which may enhance the clinical activity of subsequent therapies employing the cytotoxic potential of the T-cell system. “Subsequent treatment” may comprise further treatment with the B-cell depleting agent applied in the inventive method, with another B-cell depleting agent, further treatment as described herein or combinations thereof after the B-cell numbers have been successfully reduced to (or maintained at) one B-cell per ml/serum or less within the predefined period of time. It is in general also conceivable that no subsequent treatment is necessary. In accordance with the foregoing, the methods of the invention therefore allow stratification of patients (or groups of patients), i.e. sorting patients (or groups of patients) into those who may or may benefit from subsequent therapy. Also, it is envisaged that patients (or groups of patients) in which the number of B-cells in the blood can be reduced to or maintained at one B-cell/ml serum or less within the predefined period of time are likely to benefit from subsequent ALL therapy, while patients in which the number of B-cells in the blood exceeds one B-cell/ml serum within the predefined period of time, are not likely to benefit from subsequent therapy.

In the aforementioned method, the number of B-cells in the blood of a patient (or group of patients) having received a B-cell depleting agent is monitored/determined after a first predefined period of time after the initial treatment with said B cell depleting agent. Means and methods for determining/monitoring the number of B-cells in the blood of a patient (or group of patients) have been defined elsewhere herein. Notably, the first period of time is shorter than the predefined period of time as defined elsewhere herein, and is preferably between 2 and 14 days, i.e. 2, 3, 4, 5, 6, 7, 8, 9, 10, 11, 12, 13, or 14 days. Step (b) of the inventive method is useful in determining whether or not the patient is responsive to the administration of said B-cell depleting agent, i.e. in stratifying patients in to those likely to benefit from subsequent treatment after the first predefined period of time and those who are not. If the number of B-cells remains or falls below one B cell/ml serum within said first predefined period of time, then subsequent treatment is likely to be therapeutically effective.

If, however, the B-cell depleting agent e.g. fails to reduce or maintain the number of B-cells at the desired level of one B-cell/ml serum or less within the first predefined period of time, then the B-cell depleting agent can be adjusted such that the number of B-cells in the blood of the patient (or group of patients) remains or falls below one B cell/ml serum within a predefined period of time of preferably 15 days or less after the initial treatment with said B cell depleting agent.

In general, the first predefined period of time and the predefined period of time can be of any length, as long as the first predefined period of time is shorter than the predefined period of time. Both periods of time start with the first day of initial treatment with the B-cell depleting agent applied in the inventive methods, i.e. on the same day. The skilled practitioner will readily be able to determine the desired length of the first predefined period of time, depending on, e.g., the B-cell depleting agent, its concentration, formulation, treatment regiment, the type, the physical constitution and findings of the patient (or group of patients), and the predefined period of time.

### Adjustment

Methods of the invention may further involve “adjusting” the B-cell depleting agent. The term “adjusting” refers to an intentional modification of the B-cell depleting agent in order to achieve a number one B-cell/ml serum or less in the treated patient (or group of patients). Thus, the number of B-cells in the blood of an patient is used to adjust the dosage or treatment regimen with the B-cell depleting agent such that the number of B-cells in the blood of said patient remains or falls below one B cell/ml serum within a (first) predefined period of time after the initial treatment with said B cell depleting agent. The adjustment (intentional modification) may involve modification of the dosage, treatment regimen, formulation, or the like. For example, if the number of B-cells in the blood of a patient exceeds one B cell/ml serum within a (first) predefined period, a higher dosage of B-cell depleting agent may be administered, or the B-cell depleting agent may be administered for a prolonged period of time.

The exact dosage of B-cell depleting will depend on the purpose of the treatment (e.g. remission maintenance vs. acute flare of disease), route of administration, age, body weight, general health, sex, diet, time of administration, drug interaction and the severity of the condition, and will be ascertainable by one skilled in the art using known techniques.

Generally, any modification is conceivable as long as it preferably results in the number of B-cells in the blood of the treated patient (group of patients) being reduced to or maintained at a number of one B-cell/ml serum or less within the predefined period of time. The modification may also include additionally applying further treatments as described herein, and/or administering another B-cell depleting agent.

### Further Leukemia or Lymphoma Treatment

It is also conceivable to use further leukemia or lymphoma treatment in combination with the uses and methods of the invention. Further treatment can in general be applied antecedently, simultaneously, and/or subsequently to the uses and methods of the invention.

Hematopoietic stem cell transplantation (HSCT), for example, is a common ALL treatment. The term generally refers to transplantation of hematopoietic stem cells, usually derived from bone marrow or blood, and comprises autologous (i.e., the stem cells are derived from the patient) and allogeneic (i.e., the stem cells are derived from a donor) HSCT. For ALL treatment, allogeneic HSCT is generally preferred. It is also envisaged that the uses and methods of the present invention can be applied before or after HSCT, or both, or in between two HSCT treatments.

Patients (or groups of patients) treated according to the methods of the invention may also receive a chemotherapeutic treatment. In the context of the present invention, a “chemotherapeutic treatment” refers to a treatment with an antineoplastic agent or the combination of more than one of these agents into a standardized treatment regimen. In the context of the present invention, the term “chemotherapeutic treatment” comprises any antineoplastic agent including small sized organic molecules, peptides, oligonucleotides and the like. Agents included in the definition of chemotherapy are, without limitation, alkylating agents, e.g. mechlorethamine, cyclophosphamide, melphalan, chlorambucil, ifosfamide, busulfan, N-Nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), cisplatin, carboplatin, oxaliplatin, procarbazine and hexamethylmelamine; antimetabolites, e.g. methotrexate, pemetrexed, fluorouracil, capecitabine, cytarabine, gemcitabine, decitabine, Vidaza, fludarabine, nelarabine, cladribine, clofarabine, pentostatin, thioguanine, mercaptopurine; anti-microtubule agents e.g. vincristine, vinblastine, vinorelbine, vindesine, vinflunine, paclitaxel, docetaxel, podophyllotoxin; topoisomerase inhibitors, e.g. irinotecan, topotecan, etoposide, doxorubicin, mitoxantrone, teniposide, novobiocin, merbarone, aclarubicin; cytotoxic antibiotics, e.g. actinomycin, bleomycin, plicamycin, mitomycin, doxorubicin, daunorubicin, epirubicin, idarubicin, pirarubicin, aclarubicin, and mitoxantrone.

Further treatment also includes radiation therapy. CNS treatment or prophylaxis is also envisaged in order to prevent malignant cells from spreading in the CNS, e.g. by intrathecal chemotherapy and/or radiation therapy of the brain and spinal cord.

Since the present inventors speculate that therapeutic success of treatment could be based (in part) of an expansion of the T cell populations resulting in enhanced T-cell anti-cancer activity, inducers and enhancers of T cell activation and/or proliferation, CAR T cells, donor T cells, anti-cytotoxic T-lymphocyte-associated antigen-4 (CTLA-4) antibodies and others are also envisaged.

### Treatment

The term “treatment” in all its grammatical forms includes therapeutic or prophylactic treatment of leukemia and lymphoma, e.g. of ALL or NHL. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of leukemia and lymphoma, e.g. ALL. The term “treatment” as used herein means in the broadest sense medical procedures or applications that are intended to relieve illness. In the present case, the administration of a B-cell depleting agent (prepared for administration to a patient) as described herein is for the treatment, amelioration or elimination of leukemia or lymphoma in patients.

### Pharmaceutical Composition

It is envisaged to administer the B-cell depleting agent in the form of a pharmaceutical composition. The term “pharmaceutical composition” particularly refers to a composition suitable for administering to a human or animal, i.e., a composition containing components which are pharmaceutically acceptable. Preferably, a pharmaceutical composition comprises a B-cell depleting agent together with one or more pharmaceutical excipients. The composition may also comprise further agents as described elsewhere herein. The term “excipient” includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers or tonicity modifiers. Pharmaceutical compositions of the invention preferably comprise a therapeutically effective amount of B-cell depleting agent and can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration.

The subcutaneous formulation according to the present invention comprises, in addition to the T cell engaging polypeptide construct, potassium phosphate (preferably 10 mM), sucrose (preferably 2% (w/v)), mannitol (preferably 4% (w/v)), sulfobutylether betacyclodextrin (preferably 1% (w/v)), polysorbate 80 (preferably 0.01% (w/v)), pH 7.0.

In an alternative embodiment, the subcutaneous formulation according to the present invention comprises, in addition to the T cell engaging polypeptide construct, L-glutamic acid (preferably 10 mM), sucrose (9% (w/v)), polysorbate 80 (preferably 0.01% (w/v)), pH 4.2.

A better understanding of the present invention and of its advantages will be obtained from the following example, offered for illustrative purposes only. The example is not intended to limit the scope of the present invention in any way.

### EXAMPLES

### Example 1—A Phase 1b Study of Blinatumomab Regimen Including Subcutaneous (SC) Administration in Relapsed/Refractory (R/R) Indolent Non-Hodgkin's Lymphoma (NHL)

### Methods

Patients taking part in this study are ≥18 y with indolent NHL (follicular, marginal zone, lymphoplasmacytic, mantle cell, or small lymphocytic) that was primary refractory (1+ prior line), relapsed (within 1 y of first response), or that had responded to initial therapy for ≥1 y and relapsed after 2+ lines, including an anti-CD20 monoclonal antibody.

Patients with respective disease have not been irradiated and was measurable (≥1.5 cm) on PET-CT or CT. Patients received a 3-week continuous intravenous (cIV) run-in period followed by subcutaneous (SC) dosing in 5 cohorts, a further 2 weeks of cIV dosing, and the option for a second cycle of cIV dosing (as depicted in FIG. 1). The primary endpoint determined was safety and tolerability of SC blinatumomab; secondary endpoints included pharmacokinetics (PK), estimating the maximum tolerated dose (MTD), i.e., the highest dose at which≤1/6 pts had a dose-limiting toxicity (DLT), and efficacy (NCT 02961881).

### Results

Patients (n=29) had a median (range) age of 64 (42-75) years, 55% were male, 90% Caucasian, with follicular I-IIIA (76%), marginal zone (10%), mantle cell (10%) and lymphoplasmacytic lymphoma (3%) subtypes; no patients had prior allo-hematopoietic stem cell transplant (HSCT), 38% had prior auto-HSCT. Of the 29 patients, 5 discontinued (D/C) blinatumomab due to AEs (n=3), patient request (1), and disease progression (1); 26 patients completed the study; patients received a median (range) of 5 (3-10) doses. AEs leading to D/C in SC treatment included neurologic events of aphasia and seizure.

During SC dosing, 2 Dose-Limiting-Toxicities (DLTs) occurred (aphasia and seizure, both in 1 patient). Maximum tolerated dose (MTD) was not reached. Five patients had grade 3 AEs (thrombocytopenia, erosive esophagitis, asthenia, device-related infection, hyperglycemia, aphasia, seizure; patients may have had >1 grade ≥3 AE); there were no fatal AEs or grade 4 AEs. AEs of interest included neurologic events (all, n=15; grade ≥3, n=2), infection (2; 1), and cytokine release syndrome (4; 0). One patient had grade 1 injection site erythema. Anti-blinatumomab antibodies have not been detected.

Preliminary PK results were consistent across the cohorts and 3 different dosing regimens. Following the first dose, maximum concentrations (Cmax) were reached after ˜5-12 hours and exposures (Cmax and area under concentration-time curve [AUC] from 0-12 hours) increased in a dose-related manner. At steady state, exposures (AUC over the dosing interval) increased in a dose-related manner for dosing intervals of once every 12, 24, and 48 hours across cohorts. Blinatumomab bioavailability and apparent terminal elimination half-life were favorable for extending the dosing interval to once every other day and potentially longer intervals. Interestingly, the PK values using this administration scheme in 6 patients in the first cohort are very positive. Namely, the bioavailability of 33.7% was higher than predicted and the mean half-life was surprisingly longer than in cIV (10.8 hrs vor SC dosing versus about 2 hrs in cIV-treated patients). The steady-state concentrations during both cIV infusion periods were consistent with those previously reported in NHL patients.

In all patients, the overall response rate (ORR) per Cheson criteria was 69% (evaluable, n=23: complete response [CR], 21%; partial response [PR], 48%; cycle 1 [C1], n=22: ORR, 62%; CR, 14%; PR, 48%; cycle 2 [C2], n=17: 45%; 17%; 28%; respectively); per Lugano criteria, the ORR was 52% (n=21: CR, 24%; PR, 28%; C1, n=18: 45%; 17%; 28%; C2, n=12: 31%; 21%; 10%); for follicular lymphoma, ORR was 77% per Cheson (n=19: CR, 23%; PR, 55%) and 55% per Lugano (n=15: CR, 23%; PR, 32%). No dose dependency in efficacy or toxicity was observed because SC dosing was administered for only 1 week, following 3 weeks of cIV and prior to 2 weeks of cIV dosing; patients who did not tolerate cIV did not progress to SC dosing.

### Conclusions

In patients with R/R indolent NHL, SC blinatumomab had a favorable safety profile, with the caveat that patients who could not tolerate cIV blinatumomab did not advance to SC dosing. Efficacy was comparable with that seen for cIV dosing in prior blinatumomab NHL studies. Safety/tolerability of blinatumomab SC administration over the whole cycle is currently being evaluated in a phase 1 trial of pts with R/R acute lymphoblastic leukemia (NCT 04521231). Blinatumomab bioavailability and half-life showed promising features for SC administration, warranting further investigation.

### Example 2—Subcutaneous Administration of Blinatumomab in Patients with Relapsed/Refractory B-cell Precursor Acute Lymphoblastic Leukemia

The encouraging risk profile observed in the ongoing trial as depicted in Example 1 (ClinicalTrials.gov Identifier: NCT02961881) above and the potential convenience of an SC mode of administration induced the present inventors to investigate SC blinatumomab delivery in patients with other malignancies, such as R/R B-ALL.

### Methods

In an ongoing multicenter, single arm, open-label, phase 1b dose-finding study (NCT04521231), patients received 2-5 cycles of SC blinatumomab. Eligible patients have the following characteristics:
Age ≥18 years
Diagnosis of B-ALL
Refractory to primary induction therapy or salvage therapy
Relapsed disease including
untreated relapse (any stage)
refractory relapse, or
relapse after first salvage therapy,
relapse after allogeneic hematopoietic stem cell transplant
Eastern Cooperative Oncology Group (ECOG) performance status score ≤2
Greater than or equal to 5% blasts in the bone marrow.

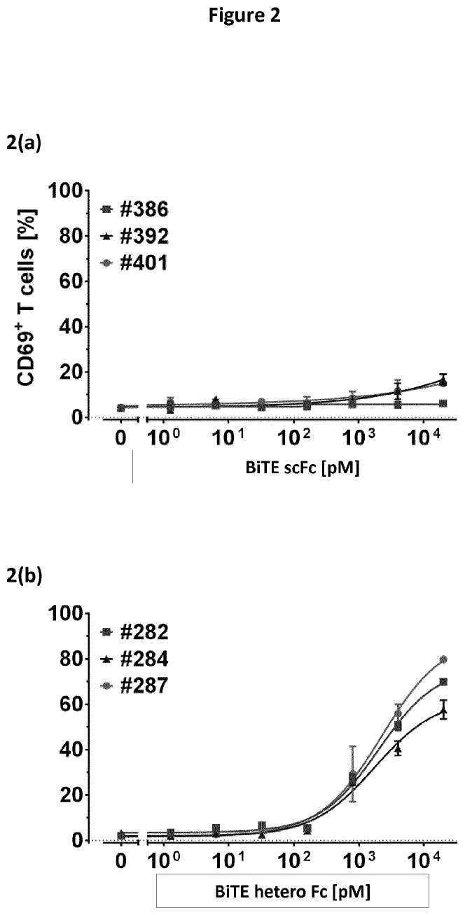

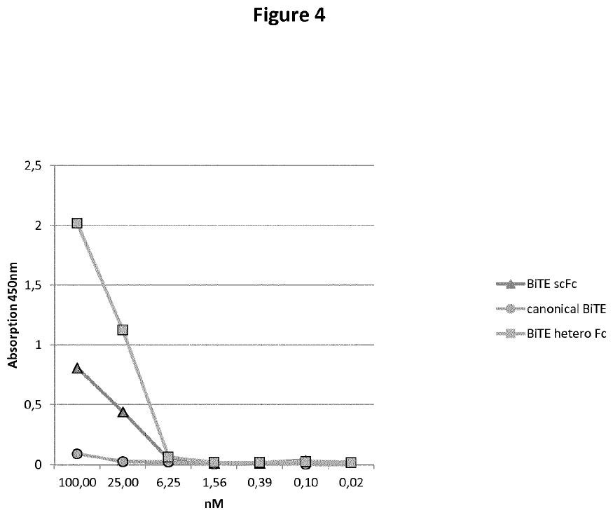

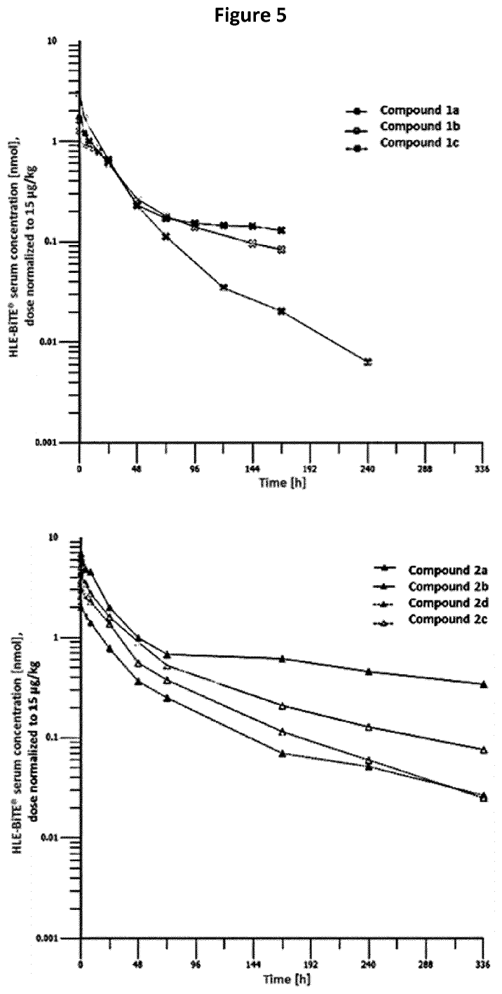

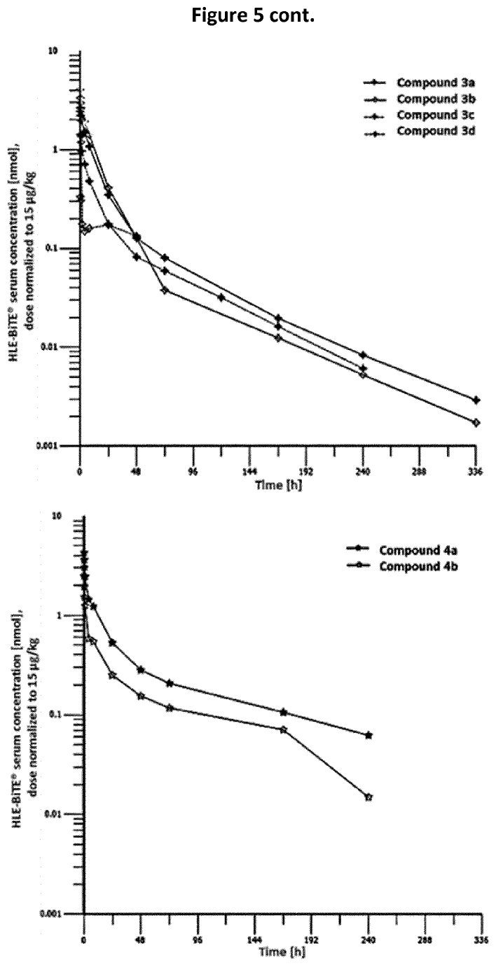

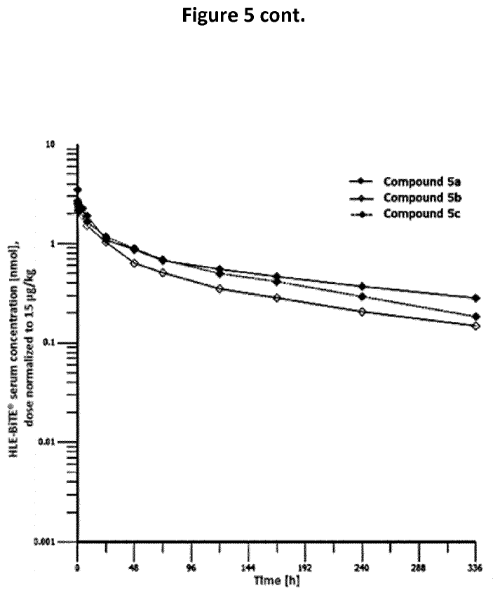

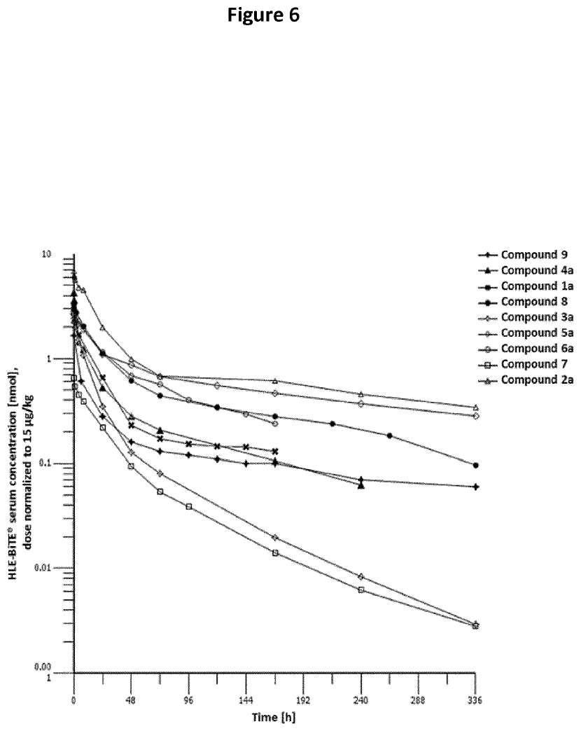

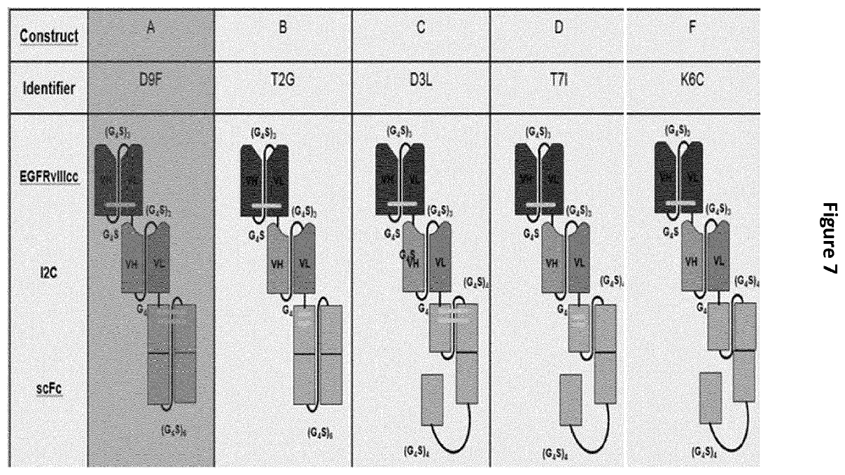

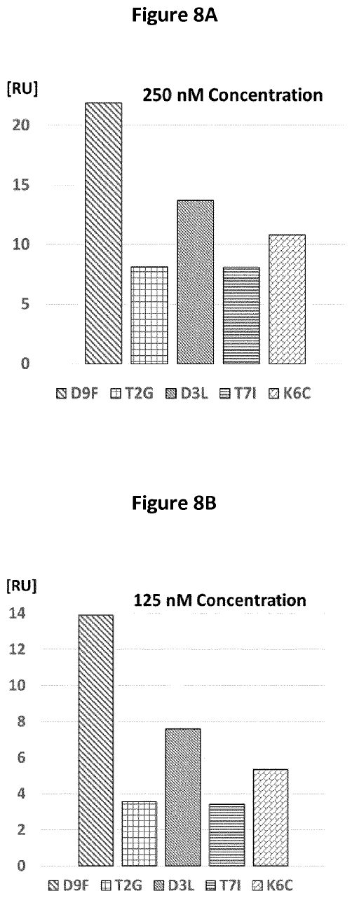

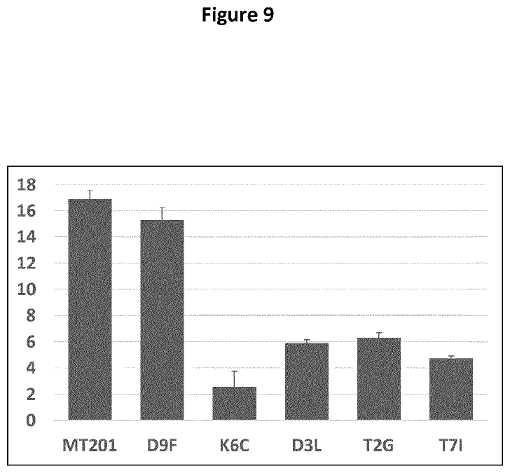

Each cycle was 34 days long and included a 26-day treatment period and an 8-day treatment-free interval. In cohort 1, cycle 1, patients received either 40 μg, 120 μg, 250 μg, 500 μg or the Maximum Tolerated Dose (MTD) 3 times weekly of SC blinatumomab once daily (QD) on days 1-7 and then 250 μg 3 times weekly (MWF) on days 8-26 in Cohorts 1 and 2, 500 μg in Cohort 3, 1000 μg in Cohort 4, or the MTD; in subsequent cycles, patients received 250 μg (Cohorts 1 and 2), 500 μg (Cohort 3), 1000 μg (Cohort 4), 3 times weekly or the MTD at 2 times weekly during the treatment period (as depicted in FIG. 2). Bone marrow (BM) evaluation was performed on day 27 of each cycle. Additional dosing regimen are shown in FIG. 4A) and B). FIG. 4A) shows an inventive dose escalation scheme that is used when patients do not show any DLT (dose-limiting toxicity) of CRS. The arrows in said figure from one cohort point to the next cohort that may be used. If a patient shows a DLT of CRS in a given cohort, a de-escalation scheme is used (as shown in FIG. 4 B)). For example, when a patient experiences a DLT of CRS in cohort 2, it is possible to de-escalate the doses according to the scheme shown in cohorts 2.1 and 2.2 in FIG. 4 B).

### Results

Six patients from cohort 1 were included in this Jun. 22, 2021 data cutoff. Median age was 64 (range 38-83) years. The number of prior therapies ranged from 2-4. Two patients had disease refractory to primary therapy or salvage therapy, 2 patients relapsed after chemotherapy, and 2 patients relapsed after prior allogeneic hematopoietic stem cell transplant. Median BM blast count at study start was 85% (range 28%-95%). Only one patient had BM blasts <50% (28%). At enrollment, all patients had an ECOG score of 0-1. The median number of SC blinatumomab cycles initiated was 1 (range 1-3).

Exposures for SC doses were comparable to the efficacious exposures of the approved cIV regimen for blinatumomab: mean steady state concentrations were 215 and 853 μg/mL for 40 μg QD and 250 μg three times weekly SC doses, respectively, vs 228 and 616 μg/mL for 9 and 28 μg/day cIV dosing, respectively. The pharmacodynamic profile following SC blinatumomab of peripheral immune cell redistribution (circulating CD3+ and CD8+ CD69+ T cells), transient cytokine elevation (IL-6, IL-10, IFN-Gamma) and CD19+ B cell counts declining below the detection limit was consistent with the historical pharmacodynamic profile for patients with cIV administration.

No grade ≥3 cytokine release syndrome events were reported (Table 1). One patient developed herpes encephalitis and experienced a grade 5 neurological event unrelated to blinatumomab; no other neurological events were reported. Two patients discontinued treatment because of adverse events (injection site reaction in patient with no response, hyperleukocytosis due to disease progression).

Three patients had complete hematological response (CR) with no measurable residual disease (MRD) (<104) within 2 cycles and 1 patient had a morphological partial response (95% BM blasts at start of cycle 1 to 22% blasts on day 15). This patient discontinued on day 15 of cycle 1 after progression of extramedullary disease. At the time of the data cutoff, 2 patients remained on study. These patients had CR with no MRD.

### Conclusions

In this study in cohort 1, SC blinatumomab has demonstrated beneficial anti-leukemia activity in heavily pretreated patients with R/R B-ALL. Pharmacokinetic and pharmacodynamic results support the use of SC dosing. The safety profile was manageable and consistent with that reported for cIV blinatumomab.

TABLE 1
Treatment-emergent Treatment-related Adverse Events and Worst Grade

| | Any | | | | | |
|---|---|---|---|---|---|---|
| | Grade | Grade 1 | Grade 2 | Grade 3 | Grade 4 | Grade 5 |
| Preferred Term | N = 6 (%) | n (%) | n (%) | n (%) | n (%) | n (%) |
| CRS | 4 (66.7) | 2 (33.3) | 2 (33.3) | 0 (0.0) | 0 (0.0) | 0 (0.0) |
| Pyrexia | 2 (33.3) | 0 (0.0) | 2 (33.3) | 0 (0.0) | 0 (0.0) | 0 (0.0) |
| Alanine | 1 (16.7) | 0 (0.0) | 0 (0.0) | 1 (16.3) | 0 (0.0) | 0 (0.0) |
| aminotransferase | | | | | | |
| increased | | | | | | |
| Aspartate | 1 (16.7) | 1 (16.7) | 0 (0.0) | 0 (0.0) | 0 (0.0) | 0 (0.0) |
| aminotransferase | | | | | | |
| increased | | | | | | |
| Asthenia | 1 (16.7) | 0 (0.0) | 1 (16.7) | 0 (0.0) | 0 (0.0) | 0 (0.0) |
| Blood bilirubin | 1 (16.7) | 0 (0.0) | 1 (16.7) | 0 (0.0) | 0 (0.0) | 0 (0.0) |
| increased | | | | | | |
| Injection site | 2 (33.3) | 1 (16.7) | 1 (16.7) | 0 (0.0) | 0 (0.0) | 0 (0.0) |
| reaction | | | | | | |
| Neutropenia | 1 (16.7) | 0 (0.0) | 0 (0.0) | 1 (16.7) | 0 (0.0) | 0 (0.0) |
| Sepsis | 1 (16.7) | 0 (0.0) | 0 (0.0) | 1 (16.7) | 0 (0.0) | 0 (0.0) |
| Thrombocytopenia | 1 (16.7) | 0 (0.0) | 0 (0.0) | 0 (0.0) | 1 (16.7) | 0 (0.0) |

During the clinical trial above, additional data, now including 9 patients, in total are summarized in table 2.

Blinatumomab serum concentrations, lymphocyte numbers, and serum cytokine concentrations were assessed before each dose and at regular intervals post-dose in treatment cycles 1 and 2 as per protocol.

### Safety Assessments

Adverse events were graded using the Common Terminology Criteria for Adverse Events (CTCAE).

### Efficacy Assessments

A complete remission (CR) is defined as
less than 5% blasts in the bone marrow
no evidence of disease
full recovery of peripheral blood counts
platelet count >100,000/μl
absolute neutrophil count >1,000/μl

A complete remission with partial hematological recovery (CRh) is defined as
less than 5% blasts in the bone marrow
no evidence of disease
partial recovery of peripheral blood counts
platelet count >50,000/μl and
absolute neutrophil count >500/μl

A complete remission with incomplete hematological recovery (CRi) is defined as
less than 5% blasts in the bone marrow
no evidence of disease and
incomplete recovery of peripheral blood counts
platelet count >100,000/μl or
absolute neutrophil count >1000/μl (but not both)

A minimal residual disease (MRD) response is defined as the detection of less than 10-4 leukemic cells detectable by flow cytometry or PCR

TABLE 2
Pharmacokinetic and pharmacodynamic assessments

| | All patients | | | | | |
|---|---|---|---|---|---|---|
| below: | N = 9 | Grade 1 | Grade 2 | Grade 3 | Grade 4 | Grade 5 |
| Adverse event | n (%) | n (%) | n (%) | n (%) | n (%) | n (%) |
| CRS | 5 (55.6) | 2 (22.2) | 3 (33.3) | 0 (0.0) | 0 (0.0) | 0 (0.0) |
| Pyrexia | 2 (22.2) | 0 (0.0) | 2 (22.2) | 0 (0.0) | 0 (0.0) | 0 (0.0) |
| Alanine | 1 (11.1) | 0 (0.0) | 0 (0.0) | 1 (11.1) | 0 (0.0) | 0 (0.0) |
| aminotransferase | | | | | | |
| increased | | | | | | |
| Neutropenia | 1 (11.1) | 0 (0.0) | 0 (0.0) | 1 (11.1) | 0 (0.0) | 0 (0.0) |
| Sepsis | 1 (11.1) | 0 (0.0) | 0 (0.0) | 1 (16.7) | 0 (0.0) | 0 (0.0) |
| Thrombocytopenia | 1 (11.1) | 0 (0.0) | 0 (0.0) | 0 (0.0) | 1 (11.1) | 0 (0.0) |

### Efficacy

Of a total of 9 patients in cohorts 1 and 2:
Five patients (5/9) achieved an MRD-negative CR/CRh/Cri, 3 patients were from cohort 1 (3/6) and 2 patients were from cohort 2 (2/3).
Three of five responders completed 2 to 5 treatment cycles.
1 responder proceeded to an alternate course of therapy;
1 responder developed a treatment-unrelated, grade 5 adverse event
All patients achieving a CR/CRh/CRi. did so within the first treatment cycle.
Two patients discontinued treatment due to disease progression; of these, one patient discontinued because of progression of extramedullary disease but had achieved a partial morphological response (A decrease in bone marrow blast count from 95% at the start of cycle 1 to 22% on day 15) at the time of treatment discontinuation.
Two patients were non-responders with persistent disease after at least one treatment cycle. Of these, one patient discontinued treatment following an adverse event (skin rash in cycle 2).

### Pharmacodynamics

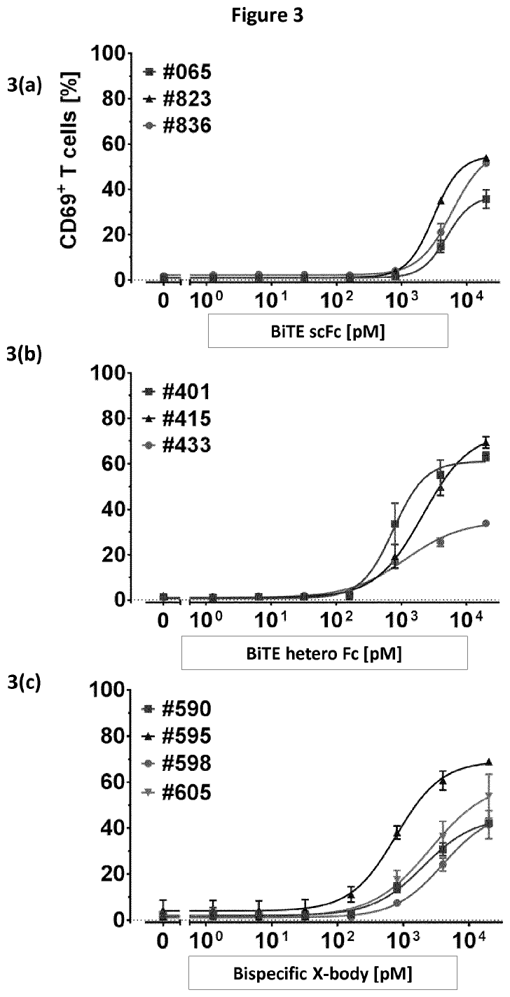

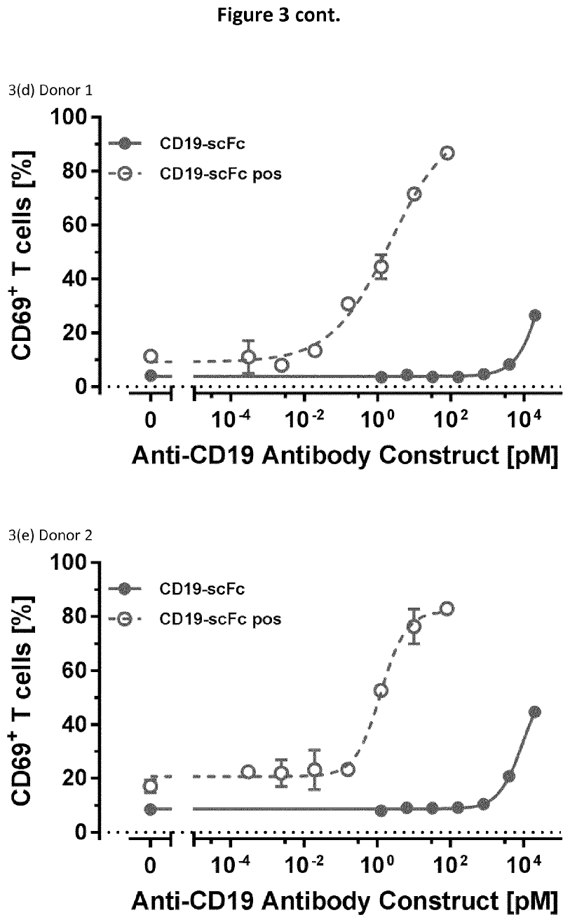

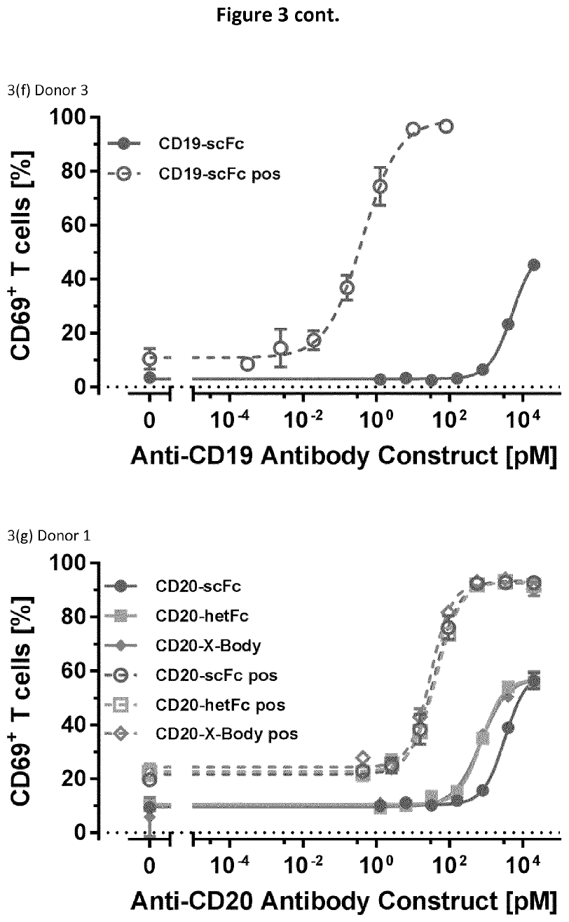

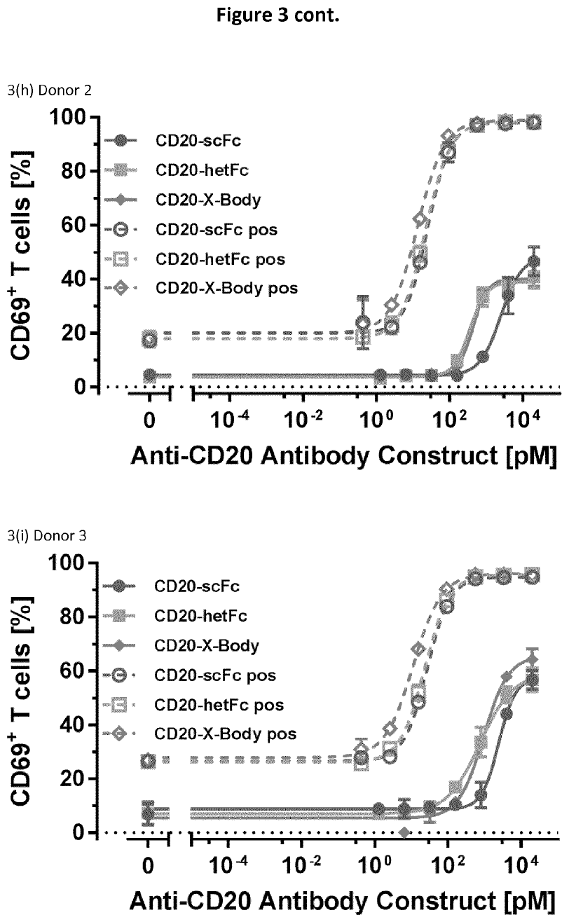

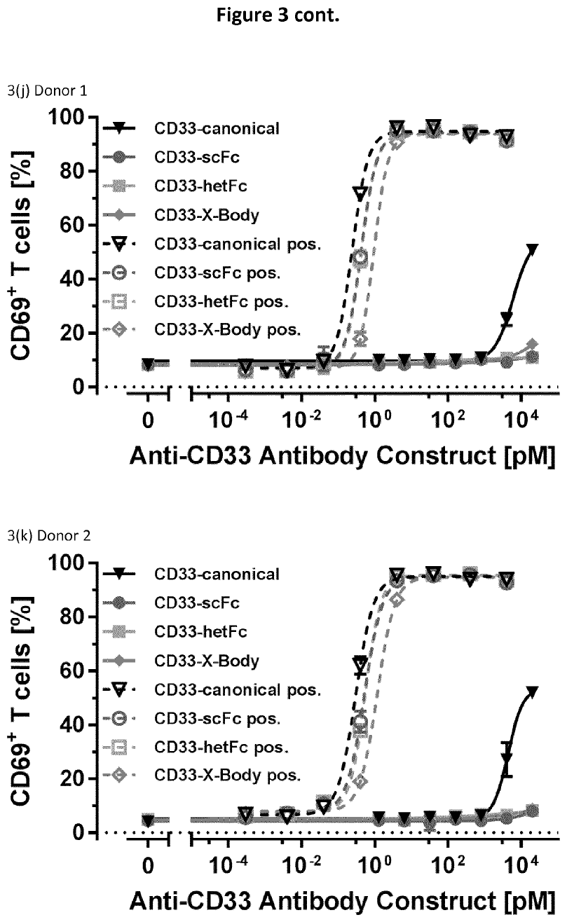

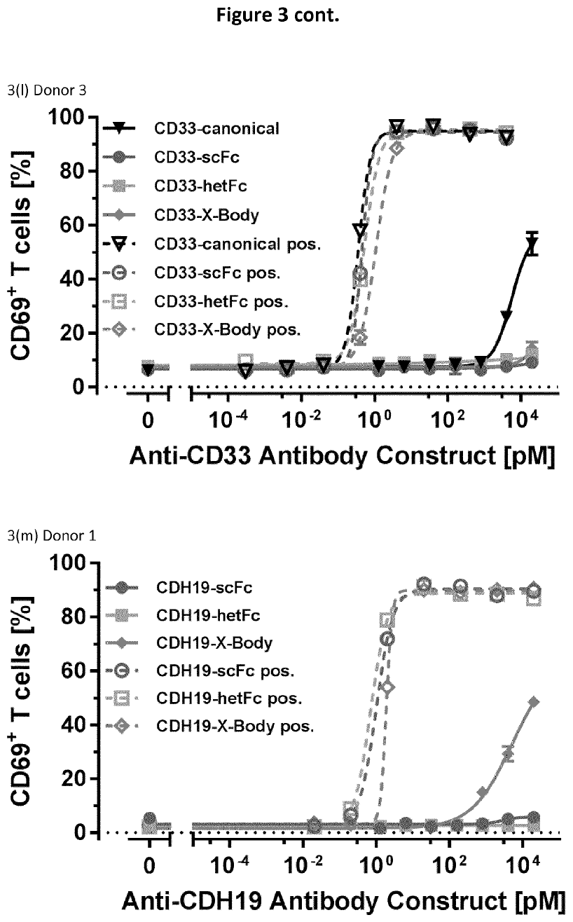

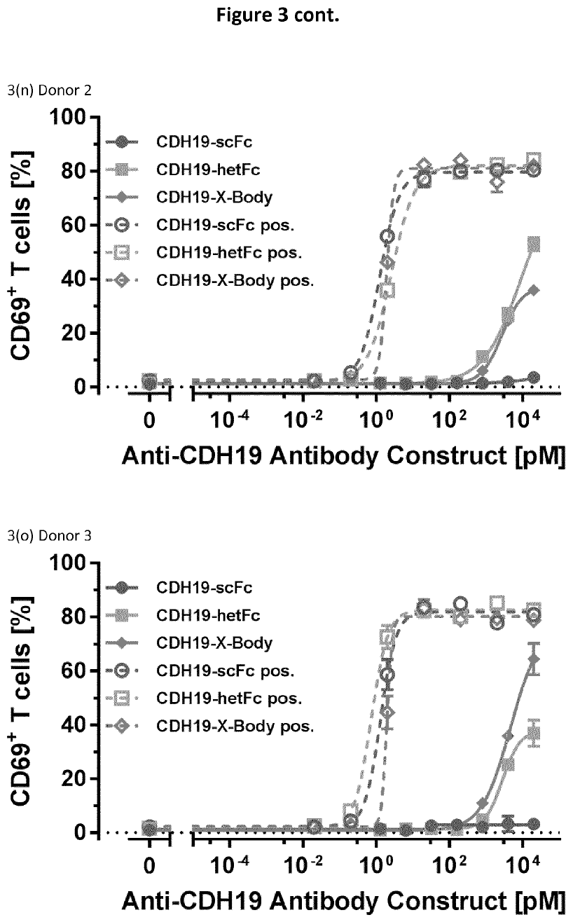

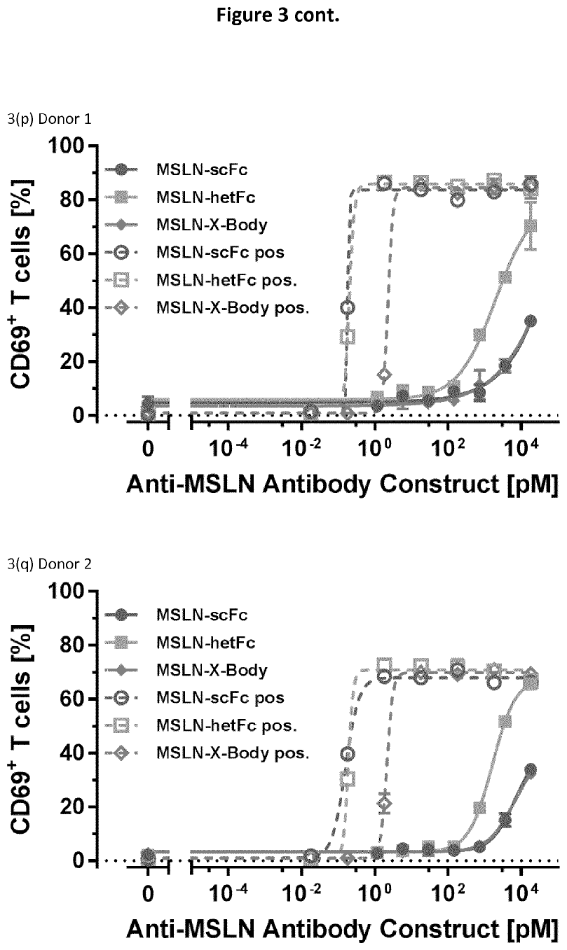

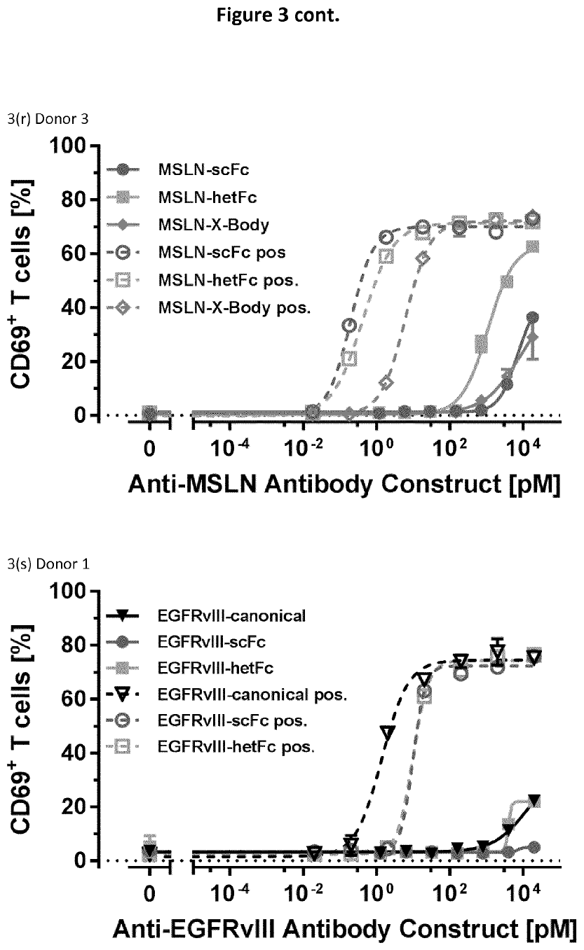

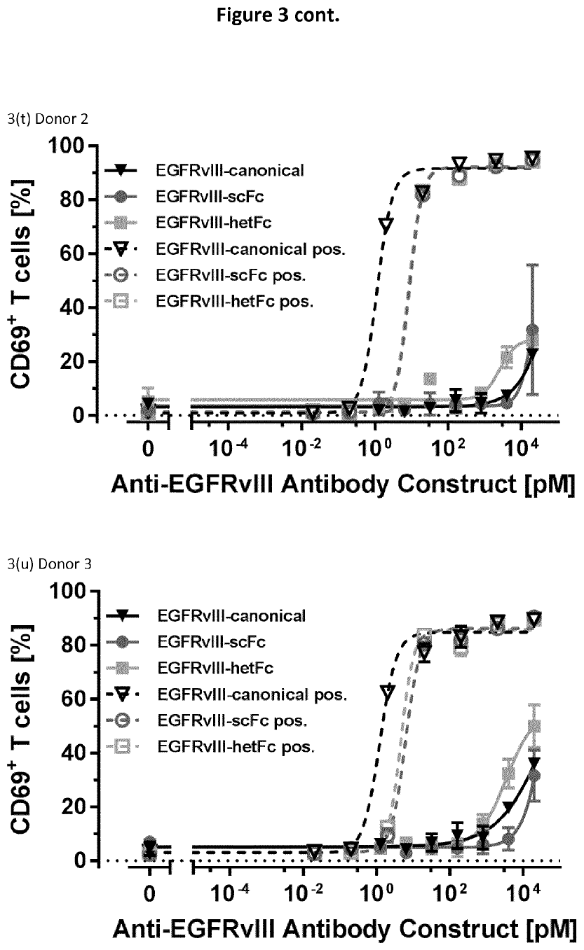

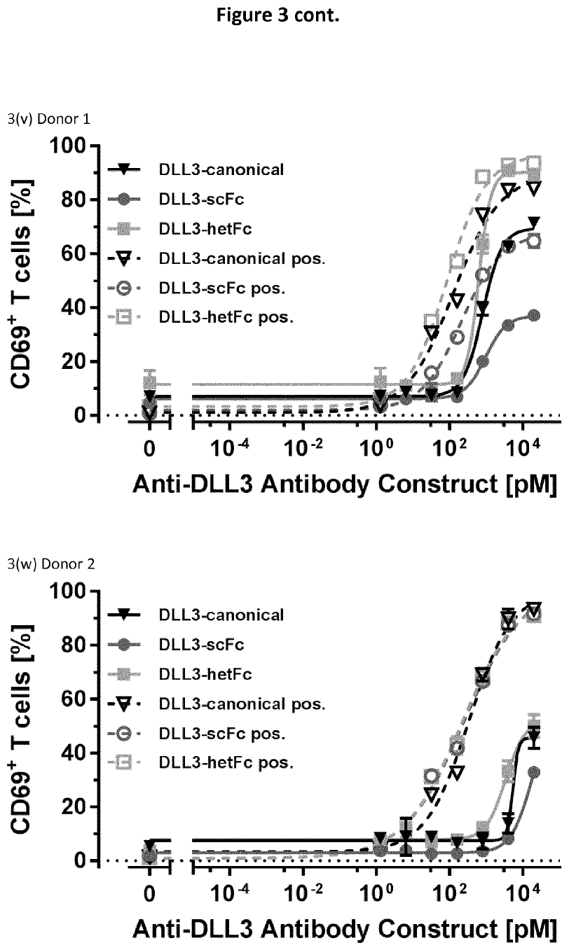

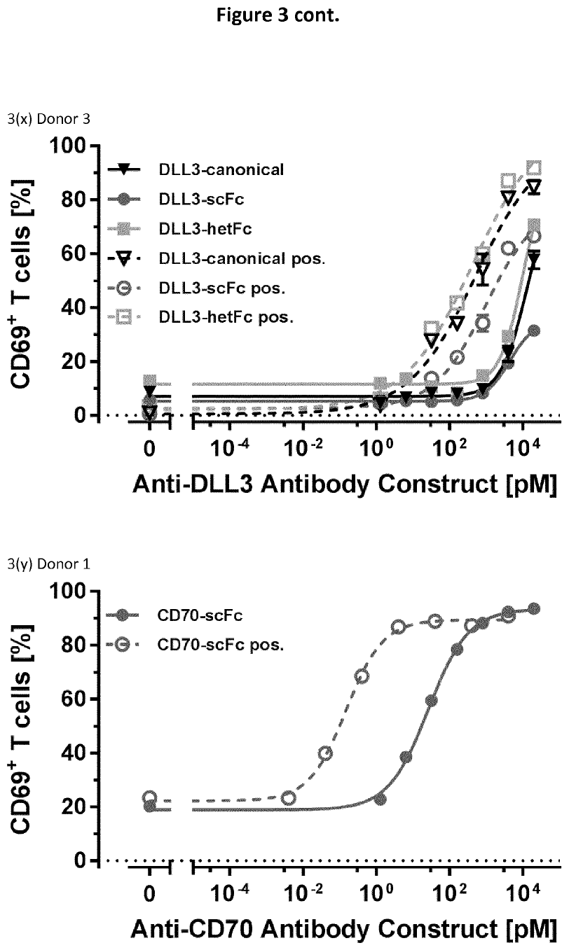

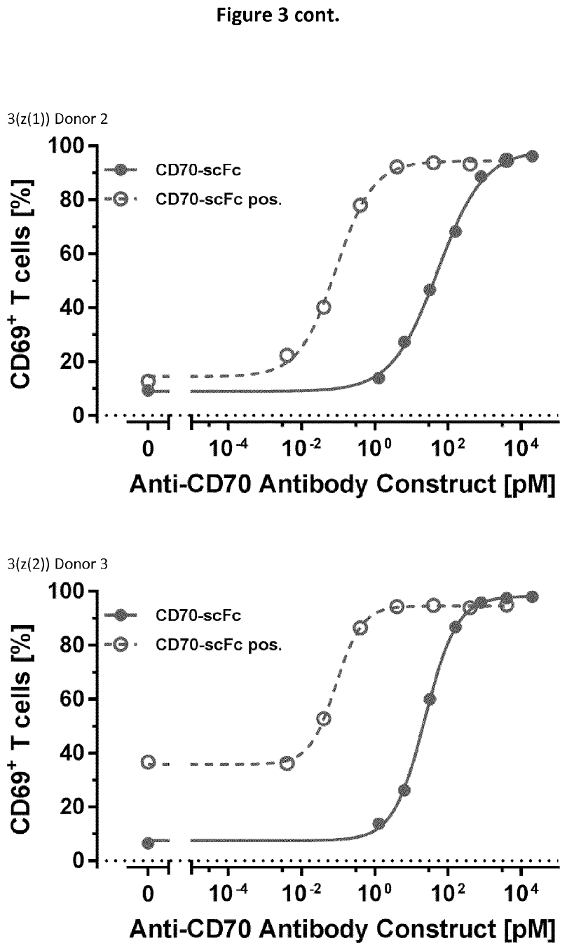

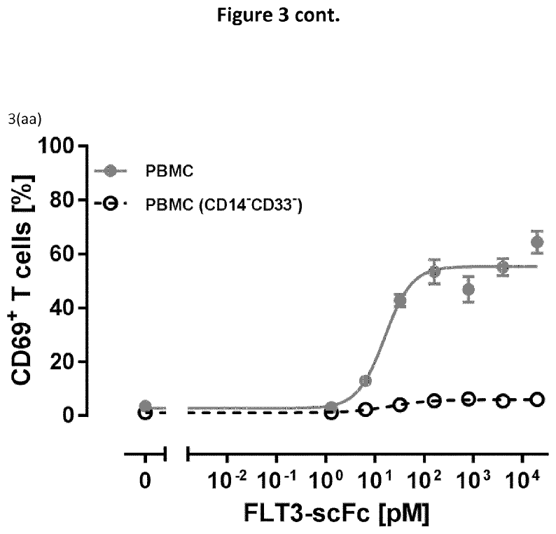

FIG. 3 shows the change in pharmacodynamic markers in response to SC blinatumomab treatment. After SC blinatumomab administration peripheral immune cell (CD3+ CD8+ CD69+ T cells) redistribution was noted within ˜ 6-30 hours (FIG. 3a, 3b). A decline in CD19+B cell numbers to below the detection limit was noted within ˜ 48 hours of start of treatment (FIG. 3c). A transient elevation in cytokine (IL-6, IL-10, IFN-γ) levels which peaked at ˜ 6-24 hours post dose was noted (FIG. 3d shows changes in IFN-γ levels following start of treatment). The pharmacodynamic profile of SC blinatumomab was similar to that observed with cIV blinatumomab. In this additional part of the analysis of results in patients receiving SC blinatumomab for R/R B-ALL. The safety profile was manageable and consistent with that reported for cIV blinatumomab. SC blinatumomab demonstrated encouraging anti-leukemic activity in heavily pretreated patients with R/R B-ALL in both tested dose levels. Evidence for similar pharmacokinetic exposures and pharmacodynamic profiles in comparison with efficacious doses of cIV blinatumomab was provided. All subjects tested thus far in cohorts 1 and 2 were negative for anti-blinatumomab antibodies. Data suggests no evidence for increased immunogenicity in the SC setting in comparison with the cIV setting.

In further cohort steps of the above-described study of the subcutaneous administration Blinatumomab to Patients with Relapsed/Refractory B-cell Precursor ALL, the efficacy of the regimen was confirmed. Out of 14 patients (64%) nine individuals experienced complete remissions within 1 cycle and 8/14 being free of Minimal Residual Disease (MRD).

In further cohorts of the study, a total of twenty-two patients were enrolled (6 in cohort 1, 3 in cohort 2, 5 in cohort 3, and 8 in cohort 4). Median age was 58 years (range, 19-83). Ten patients (50%) were aged >55 years. The number of prior therapies ranged from 2 to 4. All patients had an Eastern Cooperative Oncology Group score of 0-1 at enrollment. Six patients were refractory to frontline or salvage therapy, 9 patients relapsed after chemotherapy, 3 patients relapsed after prior hematopoietic stem cell transplantation (HSCT), 1 patient relapsed after prior HSCT and anti-CD19 chimeric antigen receptor T cell therapy, and 1 patient relapsed after 2 prior HSCTs and blinatumomab therapy where initial residual disease was cleared with cIV blinatumomab enabling first HSCT. The median number of cycles of SC blinatumomab received was 1 (range, 1-5). Seven patients received 1 cycle, 1 patient received 2, 1 patient received 4 and 2 patients received 5. Six patients ended treatment during cycle 1 and in 3 patients cycle 1 is ongoing. Median bone marrow blast count at the start of the study was 77% (range, 6-100%).

No dose-limiting toxicities were reported in any cohort. Seventeen patients had grade ≥3 treatment-emergent adverse events (TEAEs; Table). Six pts (30.0%) had neurotoxicity (NT) at any grade and 4 patients (20%) had NT at grade 3. Sixteen patients (80.0%) had cytokine release syndrome (CRS) at any grade and 2 patients (10.0%) had CRS at grade 3 (cohort 4; each event resolved within 48 h and subsequent cycle 1 dose was restarted). There was no incidence of NT or CRS at grade ≥4. Six patients ended treatment due to TEAEs, of which 4 patients were from cohort 1 (n=1, grade 5 herpes encephalitis unrelated to blinatumomab; n=1, injection site reaction in cycle 2 in patients with no response; n=1, hyperleukocytosis due to disease progression; n=1, face swelling due to progression of extramedullary disease) and 2 patients were from cohort 3 (n=1, grade 2 CRS, grade 3 liver enzyme elevation, and grade 3 NT [dysarthria and disorientation]; n=1, disease non-response). Preliminary PK results showed that observed exposures (average concentrations at steady state [SS]) of SC blinatumomab were consistent with the efficacious exposures (SS concentration) of the approved regimen of cIV blinatumomab. The PD profile demonstrated that peripheral T cell redistribution and activation (CD3+ and CD8+CD69+ T cells), transient cytokine elevation (IL-6, IL-10, IFN-γ), and CD19+ B cell depletion were consistent with the historical PD profile for cIV blinatumomab.

Nine of 14 patients (64.3%) in cohorts 1-3 achieved complete response with full or partial hematologic recovery (CR/CRh) within 2 cycles of SC blinatumomab without the presence of measurable residual disease (MRD<10⁻⁴).

In Cohort 4, eight patients were enrolled. Several weeks after the initiation of Cohort 4, and as observed in Cohorts 1-3, the subjects in this clinical study are negative for anti-blinatumomab antibodies. There is also no increased immunogenicity in the subcutaneous setting compared with continuous intravenous (cIV) administration. Current anti-drug antibody (ADA) rate in this study is consistent with the historical ADA rate to blinatumomab (cIV) across several clinical studies, and aggregate data across dose escalation shows no evidence for immunogenicity to subcutaneously administered blinatumomab. Further, in cohort 4 (500 μg subcutaneous on days 1-7, i.e., week 1; 1000 μg subcutaneously administered 3 times weekly in weeks 2 to 4) no dose-limiting toxicities (DLTs) have been observed. Of the 7/8 DLT evaluable subjects (early September 2022), 5 completed at least 1 cycle, and 5/7 (71%) DLT-evaluable subjects showed complete remissions (MRD negative). No clinically significant vital sign findings and laboratory findings (e.g., haematological, chemistry, coagulation, urinalysis, CSF analysis) were noted.

In this ongoing phase 1b dose-escalation study, SC blinatumomab demonstrated an acceptable safety profile and anti-leukemia activity in heavily pretreated patients with R/R B-ALL. PK exposures and PD profiles were consistent with those reported for the cIV regimen of blinatumomab, supporting the use of SC dosing of blinatumomab in this patient population. Encouraging PK results from SC dosing of blinatumomab through cohort 4 have been obtained.
Cₘₐₓ reached with median of 6-12 hrs.
Approximately dose proportional increase in exposure
   QD dosing: 10- and 12-fold increases in mean Cmax and AUC, respectively, for a 12.5-fold-increase in dose from 40 μg to 500 μg following first dose on day 1. Similar results with day 4 dosing at steady state.
   TIW dosing: 3.1- and 3.2-fold increases in mean Cmax and AUC, respectively, for a 4-fold increase in dose from 250 μg to 1000 μg at steady state following day 10 dosing.
   Observed apparent half-life (˜8-19 h) is consistent with SC administration in NHL subjects.
   SC bioavailability estimates consistent with SC administration in NHL subjects: approx. 27% (cross-study comparison to cIV using CL of 1.89 L/hr calculated from historical mean Css value of 616 μg/mL for 28 μg/d cIV dosing from other R/R ALL studies).

TABLE 3
TEAEs in patients with R/R B-ALL treated with SC blinatumomab

| | Total | Cohort 1 | Cohort 2 | Cohort 3 | Cohort 4 |
|---|---|---|---|---|---|
| | (N = 20) | (N = 6) | (N = 3) | (N = 5) | (N = 8) |
| | n (%) | n (%) | n (%) | n (%) | n (%) |
| TEAEs (any grade) | 20 (100.0) | 6 (100.0) | 3 (100.0) | 5 (100.0) | 8 (100.0) |
| Grade ≥3 TEAEs | 17 (85.0) | 6 (100.0) | 2 (66.7) | 4 (80.0) | 2 (25.0) |
| Neurotoxicity | 4 (20.0) | 1 (16.7) | 0 (0.0) | 1 (20.0) | 4 (50.0) |
| Neutropenia | 3 (15.0) | 1 (16.7) | 0 (0.0) | 1 (20.0) | nd |
| Thrombocytopenia | 3 (15.0) | 2 (33.3) | 0 (0.0) | 0 (0.0) | nd |
| CRS | 2 (10.0) | 0 (0.0) | 0 (0.0) | 0 (0.0) | nd |
| Serious TEAEs | 15 (75.0) | 4 (66.7) | 2 (66.7) | 5 (100.0) | nd |

R/R B-ALL, relapsed or refractory B cell precursor acute lymphoblastic leukemia; SC, subcutaneous; TEAE, treatment-emergent adverse event; nd, not determined.

TABLE 4
Safety, clinical response data summary

| | Cohort 1 | Cohort 2 | Cohort 3 | Cohort 4 |
|---|---|---|---|---|
| | 40 μg QD / | 120 μg QD / | 250 μg QD / | 500 μg QD / |
| | 250 μg TIW | 250 μg TIW | 500 μg TIW | 1000 μg TIW |
| | (N = 6) | (N = 3) | (N = 5) | (N = 8) |
| Cohort status | Completed | Completed | Completed | Completed |
| DLTs | No | No | No | No |
| Safety (adverse event | Gr 2 CRS | Gr 2 CRS | Gr 2 CRS | Gr 3 CRS |
| and grade)^{a} | (2 of 6) | (1 of 3) | (2 of 3) | (2 of 8)^{b}; |
| | | | | Gr 2 CRS |
| | | | | (2 of 8) |
| | Gr 3 NT | Gr 2 NT | Gr 3 NT | Gr 3 NT |
| | (1 of 6; | (1 of 3; | (1 of 5; | (4 of 8; |
| | started | started | started | started |
| | Wk 3) | on D 9) | after | on Wk 1 |
| | | | 3^{rd} dose) | [2 of 8] |
| | | | | or Wk 3 |
| | | | | [2 of 8])^{b} |
| Dose interruptions | 2 of 6 | 1 of 3 | 2 of 5 | 6 of 8 |
| (in first week) | (1 of 6) | (1 of 3) | (2 of 5) | (5 of 8) |
| Clinical response | MRD- | MRD- | MRD- | MRD- |
| | negative | negative | negative | negative |
| | CR | CR | CR | CR |
| | (3 of 6, 50%) | (2 of 3, 67%) | (4 of 5, 80%) | (5 of 7, 71%) |
| | | | | (1 NE subject) |
| Immunogenicity | 3 (15.0) | 2 (33.3) | 0 (0.0) | 0 (0.0) |
| (results post baseline | | | | |
| with positive ADA) | | | | |

NT = Neurotoxicity NE non evaluable.
^{a}CRS occurred within first 3 days of treatment.
^{b}One Cohort 4 subject experienced a Gr 3 CRS (after D 1 dose) and Gr 3 NT (after 5th TIW dose)

SEQUENCES

| SEQ | protein/ | | |
|---|---|---|---|
| ID | nucleic | | |
| NO: | acid | Description | Sequence |
| 1 | protein | CD19 x CD3 | DIQLTQSPASLAVSLGQRATISCKASQSVDYDGDSYLNWYQQIPGQPPKLL |
| | | bispecfic | IYDASNLVSGIPPRFSGSGSGTDFTLNIHPVEKVDAATYHCQQSTEDPWTF |
| | | single- | GGGTKLEIKGGGGSGGGGSGGGGSQVQLQQSGAELVRPGSSVKISCKASGY |
| | | chain | AFSSYWMNWVKQRPGQGLEWIGQIWPGDGDTNYNGKFKGKATLTADESSST |
| | | molecule | AYMQLSSLASEDSAVYFCARRETTTVGRYYYAMDYWGQGTTVTVSSGGGGS |
| | | | DIKLQQSGAELARPGASVKMSCKTSGYTFTRYTMHWVKQRPGQGLEWIGYI |
| | | | NPSRGYTNYNQKFKDKATLTTDKSSSTAYMQLSSLTSEDSAVYYCARYYDD |
| | | | HYCLDYWGQGTTLTVSSVEGGSGGSGGSGGSGGVDDIQLTQSPAIMSASPG |
| | | | EKVTMTCRASSSVSYMNWYQQKSGTSPKRWIYDTSKVASGVPYRFSGSGSG |
| | | | TSYSLTISSMEAEDAATYYCQQWSSNPLTFGAGTKLELK |
| 2 | Nucleic | CD19 x CD3 | GATATCCAGCTGACCCAGTCTCCAGCTTCTTTGGCTGTGTCTCTAGGGCAG |
| | acid | bispecfic | AGGGCCACCATCTCCTGCAAGGCCAGCCAAAGTGTTGATTATGATGGTGAT |
| | | single- | AGTTATTTGAACTGGTACCAACAGATTCCAGGACAGCCACCCAAACTCCTC |
| | | chain | ATCTATGATGCATCCAATCTAGTTTCTGGGATCCCACCCAGGTTTAGTGGC |
| | | molecule | AGTGGGTCTGGGACAGACTTCACCCTCAACATCCATCCTGTGGAGAAGGTG |
| | | | GATGCTGCAACCTATCACTGTCAGCAAAGTACTGAGGATCCGTGGACGTTC |
| | | | GGTGGAGGGACCAAGCTCGAGATCAAAGGTGGTGGTGGTTCTGGCGGCGGC |
| | | | GGCTCCGGTGGTGGTGGTTCTCAGGTGCAGCTGCAGCAGTCTGGGGCTGAG |
| | | | CTGGTGAGGCCTGGGTCCTCAGTGAAGATTTCCTGCAAGGCTTCTGGCTAT |
| | | | GCATTCAGTAGCTACTGGATGAACTGGGTGAAGCAGAGGCCTGGACAGGGT |
| | | | CTTGAGTGGATTGGACAGATTTGGCCTGGAGATGGTGATACTAACTACAAT |
| | | | GGAAAGTTCAAGGGTAAAGCCACTCTGACTGCAGACGAATCCTCCAGCACA |
| | | | GCCTACATGCAACTCAGCAGCCTAGCATCTGAGGACTCTGCGGTCTATTTC |
| | | | TGTGCAAGACGGGAGACTACGACGGTAGGCCGTTATTACTATGCTATGGAC |
| | | | TACTGGGGCCAAGGGACCACGGTCACCGTCTCCTCCGGAGGTGGTGGATCC |
| | | | GATATCAAACTGCAGCAGTCAGGGGCTGAACTGGCAAGACCTGGGGCCTCA |
| | | | GTGAAGATGTCCTGCAAGACTTCTGGCTACACCTTTACTAGGTACACGATG |
| | | | CACTGGGTAAAACAGAGGCCTGGACAGGGTCTGGAATGGATTGGATACATT |
| | | | AATCCTAGCCGTGGTTATACTAATTACAATCAGAAGTTCAAGGACAAGGCC |
| | | | ACATTGACTACAGACAAATCCTCCAGCACAGCCTACATGCAACTGAGCAGC |
| | | | CTGACATCTGAGGACTCTGCAGTCTATTACTGTGCAAGATATTATGATGAT |
| | | | CATTACTGCCTTGACTACTGGGGCCAAGGCACCACTCTCACAGTCTCCTCA |
| | | | GTCGAAGGTGGAAGTGGAGGTTCTGGTGGAAGTGGAGGTTCAGGTGGAGTC |
| | | | GACGACATTCAGCTGACCCAGTCTCCAGCAATCATGTCTGCATCTCCAGGG |
| | | | GAGAAGGTCACCATGACCTGCAGAGCCAGTTCAAGTGTAAGTTACATGAAC |
| | | | TGGTACCAGCAGAAGTCAGGCACCTCCCCCAAAAGATGGATTTATGACACA |
| | | | TCCAAAGTGGCTTCTGGAGTCCCTTATCGCTTCAGTGGCAGTGGGTCTGGG |
| | | | ACCTCATACTCTCTCACAATCAGCAGCATGGAGGCTGAAGATGCTGCCACT |
| | | | TATTACTGCCAACAGTGGAGTAGTAACCCGCTCACGTTCGGTGCTGGGACC |
| | | | AAGCTGGAGCTGAAA |
| 3 | protein | VH anti | QVQLQQSGAELVRPGSSVKISCKASGYAFSSYWMNWVKQRPGQGLEWIGQI |
| | | CD19 | WPGDGDTNYNGKFKGKATLTADESSSTAYMQLSSLASEDSAVYFCARRETT |
| | | | TVGRYYYAMDYWGQGTTVTVSS |
| 4 | Nucleic | VH anti | CAGGTGCAGCTGCAGCAGTCTGGGGCTGAGCTGGTGAGGCCTGGGTCCTCA |
| | acid | CD19 | GTGAAGATTTCCTGCAAGGCTTCTGGCTATGCATTCAGTAGCTACTGGATG |
| | | | AACTGGGTGAAGCAGAGGCCTGGACAGGGTCTTGAGTGGATTGGACAGATT |
| | | | TGGCCTGGAGATGGTGATACTAACTACAATGGAAAGTTCAAGGGTAAAGCC |
| | | | ACTCTGACTGCAGACGAATCCTCCAGCACAGCCTACATGCAACTCAGCAGC |
| | | | CTAGCATCTGAGGACTCTGCGGTCTATTTCTGTGCAAGACGGGAGACTACG |
| | | | ACGGTAGGCCGTTATTACTATGCTATGGACTACTGGGGCCAAGGGACCACG |
| | | | GTCACCGTCTCCTCC |
| 5 | protein | VL anti | DIQLTQSPASLAVSLGQRATISCKASQSVDYDGDSYLNWYQQIPGQPPKLL |
| | | CD19 | IYDASNLVSGIPPRFSGSGSGTDFTLNIHPVEKVDAATYHCQQSTEDPWTF |
| | | | GGGTKLEIK |
| 6 | Nucleic | VL anti | GATATCCAGCTGACCCAGTCTCCAGCTTCTTTGGCTGTGTCTCTAGGGCAG |
| | acid | CD19 | AGGGCCACCATCTCCTGCAAGGCCAGCCAAAGTGTTGATTATGATGGTGAT |
| | | | AGTTATTTGAACTGGTACCAACAGATTCCAGGACAGCCACCCAAACTCCTC |
| | | | ATCTATGATGCATCCAATCTAGTTTCTGGGATCCCACCCAGGTTTAGTGGC |
| | | | AGTGGGTCTGGGACAGACTTCACCCTCAACATCCATCCTGTGGAGAAGGTG |
| | | | GATGCTGCAACCTATCACTGTCAGCAAAGTACTGAGGATCCGTGGACGTTC |
| | | | GGTGGAGGGACCAAGCTCGAGATCAAA |
| 7 | protein | VH anti CD3 | DIKLQQSGAELARPGASVKMSCKTSGYTFTRYTMHWVKQRPGQGLEWIGYI |
| | | | NPSRGYTNYNQKFKDKATLTTDKSSSTAYMQLSSLTSEDSAVYYCARYYDD |
| | | | HYCLDYWGQGTTLTVSS |
| 8 | Nucleic | VH anti CD3 | GATATCAAACTGCAGCAGTCAGGGGCTGAACTGGCAAGACCTGGGGCCTCA |
| | acid | | GTGAAGATGTCCTGCAAGACTTCTGGCTACACCTTTACTAGGTACACGATG |
| | | | CACTGGGTAAAACAGAGGCCTGGACAGGGTCTGGAATGGATTGGATACATT |
| | | | AATCCTAGCCGTGGTTATACTAATTACAATCAGAAGTTCAAGGACAAGGCC |
| | | | ACATTGACTACAGACAAATCCTCCAGCACAGCCTACATGCAACTGAGCAGC |
| | | | CTGACATCTGAGGACTCTGCAGTCTATTACTGTGCAAGATATTATGATGAT |
| | | | CATTACTGCCTTGACTACTGGGGCCAAGGCACCACTCTCACAGTCTCCTCA |
| 9 | protein | VL anti CD3 | DIQLTQSPAIMSASPGEKVTMTCRASSSVSYMNWYQQKSGTSPKRWIYDTS |
| | | | KVASGVPYRFSGSGSGTSYSLTISSMEAEDAATYYCQQWSSNPLTFGAGTK |
| | | | LELK |
| 10 | Nucleic | VL anti CD3 | GACATTCAGCTGACCCAGTCTCCAGCAATCATGTCTGCATCTCCAGGGGAG |
| | acid | | AAGGTCACCATGACCTGCAGAGCCAGTTCAAGTGTAAGTTACATGAACTGG |
| | | | TACCAGCAGAAGTCAGGCACCTCCCCCAAAAGATGGATTTATGACACATCC |
| | | | AAAGTGGCTTCTGGAGTCCCTTATCGCTTCAGTGGCAGTGGGTCTGGGACC |
| | | | TCATACTCTCTCACAATCAGCAGCATGGAGGCTGAAGATGCTGCCACTTAT |
| | | | TACTGCCAACAGTGGAGTAGTAACCCGCTCACGTTCGGTGCTGGGACCAAG |
| | | | CTGGAGCTGAAA |
| 11 | protein | CD3 CDR-H1 | GYTFTRYTMH |
| 12 | protein | CD3 CDR-H2 | YINPSRGYTNYNQKFKD |
| 13 | protein | CD3 CDR-H3 | YYDDHYCLDY |
| 14 | protein | CD3 CDR-L1 | RASSSVSYMN |
| 15 | protein | CD3 CDR-L2 | DTSKVAS |
| 16 | protein | CD3 CDR-L3 | QQWSSNPLT |
| 17 | protein | CD19 CDR-H1 | GYAFSSYWMN |
| 18 | protein | CD19 CDR-H2 | QIWPGDGDTNYNGKFKG |
| 19 | protein | CD19 CDR-H3 | RETTTVGRYYYAMDY |
| 20 | protein | CD19 CDR-L1 | KASQSVDYDGDSYLN |
| 21 | protein | CD19 CDR-L2 | DASNLVS |
| 22 | protein | CD19 CDR-L3 | QQSTEDPWT |

### REFERENCES CITED

1. WO 99/54440
2. https://www.ema.europa.eu/en/medicines/human/EPAR/blincyto
3. WO 2004/106381
4. Mack, J. Immunol. (1997), 158, 3965-70;
5. Mack, PNAS, (1995), 92, 7021-5;
6. Kufer, Cancer Immunol. Immunother., (1997), 45, 193-7;
7. Löffler, Blood, (2000), 95, 6, 2098-103;
8. Brühl, J. Immunol., (2001), 166, 2420-2426.
9. Sela, (1969) Science 166, 1365
10. Laver, (1990) Cell 61, 553-6

1. A method of treating or ameliorating a leukemia in a patient using a T cell engaging polypeptide construct binding to CD19 and comprising the steps:
administering the T cell engaging polypeptide construct to the patient in a first treatment cycle,
wherein at least two individual doses of a first quantity of the T cell engaging polypeptide construct are subcutaneously administered in a first predetermined period; and
optionally wherein at least two individual doses of a second quantity of the T cell engaging polypeptide construct are subcutaneously administered in a second predetermined period, and
wherein the T cell engaging polypeptide construct comprises complementarity-determining regions comprising the amino acid sequences of SEQ ID NOs: 11-22.

2. The method according to claim 1, wherein the first treatment cycle is preceded by and/or followed by at least one additional treatment cycle.

3. The method according to claim 1, wherein a third predetermined treatment-free period precedes and/or follows the first treatment cycle.

4. The method according to claim 1, wherein a fourth predetermined treatment-free period follows the first predetermined period.

5. The method according to claim 1, wherein the first quantity is administered in 2 to 9 individual doses.

6. The method according to claim 1, wherein the first predetermined period is 5 to 9 days, particularly 7 days.

7. The method according to claim 1, wherein the first quantity is 10 to 80 μg, 20 to 80 μg, particularly 30 to 75 μg, more particularly 35 to 50 μg, more particularly 40 μg.

8. The method according to claim 1, wherein the first quantity is 100 to 150 μg, 110 μg to 130 μg, particularly 120 g.

9. The method according to claim 1, wherein the first quantity is 150 to 300 μg, 200 μg to 275 μg, particularly 250 g.

10. The method according to claim 1, wherein the first quantity is 250 to 750 μg, 375 μg to 625 μg, particularly 500 μg.

11. The method according to claim 1, wherein the first quantity is 500 to 1500 μg, 600 μg to 1250 μg, particularly 1000 μg.

12. The method according to claim 1, wherein the second predetermined period is 1 to 28 days, particularly 1 to 21 days.

13. The method according to claim 1, wherein the second quantity is 200 to 750 μg, particularly 225 to 275 μg, more particularly 250 μg.

14. The method according to claim 1, wherein the second quantity is 250 to 750 μg, 375 μg to 625 μg, particularly 500 g.

15. The method according to claim 1, wherein the second quantity is 500 to 1250 μg, 600 μg to 1250 μg, particularly 1000 μg.

16. The method according to claim 1, wherein the second quantity is administered 2 to 5 times weekly, particularly 3 times weekly.

17. The method according to claim 1, wherein the according to claim 1, wherein quantity administered in the at least one subsequent cycle is 200 to 300 μg, particularly 225 to 275 μg, more particularly 250 μg.

18. The method according to claim 1, wherein the quantity administered in the at least one subsequent cycle is 250 to 750 μg, 375 μg to 625 μg, particularly 500 μg.

19. The method according to claim 1, wherein the quantity administered in the at least one subsequent cycle is 500 to 1500 μg, 750 μg to 1250 μg, particularly 1000 μg.

20. The method according to claim 1, wherein the quantity in the at least one subsequent cycle is administered 2 to 5 times weekly, preferably 3 times weekly, which optionally is administered subcutaneously.

21. The method according to claim 1, wherein the quantity in the at least one subsequent cycle is administered 2 to 5 times weekly, preferably 2 times weekly, which optionally is administered subcutaneously

22. The method according to claim 1, wherein the first quantity is either 100 μg to 800 μg, 200 to 700 μg, particularly about 112 μg, 225 μg, 450 μg, or 675 μg.

23. The method according to claim 1, wherein the T cell engaging polypeptide construct binds to CD3.

24. The method according to claim 1, wherein the T cell engaging polypeptide construct binds to human and macaque CD3.

25. The method according to claim 1, wherein the T cell engaging polypeptide construct is a single chain polypeptide construct.

26. (canceled)

27. The method according to claim 1, wherein the T cell engaging polypeptide construct comprises the VH and VL regions depicted in SEQ ID NOs: 3, 5, 7, and 9.

28. The method according to claim 1, wherein the lymphoma or leukemia is selected from the group comprising NHL and ALL.

29. The method according to claim 1, wherein the patient has been subjected to and/or will be subjected to at least one further treatment preceding the subcutaneous administration scheme set forth in any of the preceding claims, and wherein the preceding and/or further treatment is selected from the group comprising cIV administration of a B-cell depleting agent, particularly of blinatumomab, administration with a CD19-specific CAR T-cell therapy, administration of a CD20-targeting agent and/or a chemotherapy.

30. The method according to claim 1, wherein the first quantity is 30 to 50 μg, particularly 40 μg and the patient suffers from (relapsed/refractory) B-cell precursor acute lymphoblastic leukemia.

31. The method according to claim 1, wherein the patient suffers from (relapsed/refractory, R/R) indolent Non-Hodgkin's Lymphoma.

32-40. (canceled)

41. The method according to claim 1 further comprising subcutaneously administering a pharmaceutical composition comprising a) a T cell engaging polypeptide construct, b) potassium phosphate, c) sucrose, d) mannitol, e) sulfobutylether betacyclodextrin, and f) polysorbate 80.

42. The method according to claim 41, wherein the pH of the pharmaceutical composition ranges from pH 6.5 to 7.5, pH 6.7 to 7.3, pH 6.8 to 7.2, pH 6.9 to 7.1, or is pH 7.0.

43. The method according to claim 1 further comprising subcutaneously administering a pharmaceutical composition comprising a T cell engaging polypeptide construct, L-glutamic acid, sucrose, and polysorbate 80.

44. The method according to claim 43, wherein the pH of the pharmaceutical composition ranges from pH 4.0 to 4.4, pH 4.1 to 4.3, or is pH 4.2.

45. The method according to claim 1 further comprising subcutaneously administering a pharmaceutical composition comprising a) a T cell engaging polypeptide construct, and b) 10 mM potassium phosphate, 2% (w/v) sucrose, 4% (w/v) mannitol, 1% (w/v) sulfobutylether betacyclodextrin, and 0.01% (w/v) polysorbate 80, wherein the composition has a pH of about 7.0.

46. The method according to claim 1 further comprising subcutaneously administering a pharmaceutical composition a) a T cell engaging polypeptide construct, and b) 10 mM L-glutamic acid, 9% (w/v) sucrose, 0.01% (w/v) polysorbate 80, wherein the composition has a pH of about 4.2.


SEQUENCE LISTING (22 sequences)

SEQ ID NO: 1 - protein, 498 aa, synthetic construct
  REGION 1..498: note = Synthetic
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - DNA, 1494 bp, synthetic construct
  source 1..1494: mol_type = other DNA
  misc_feature 1..1494: note = Synthetic
gatatccagctgacccagtctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctcgagatcaaaggtggtggtggttctggcggcggcggctccggtggtggtggttctcaggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggccaagggaccacggtcaccgtctcctccggaggtggtggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 3 - protein, 124 aa, synthetic construct
  REGION 1..124: note = Synthetic
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 4 - DNA, 372 bp, synthetic construct
  source 1..372: mol_type = other DNA
  misc_feature 1..372: note = Synthetic
caggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggccaagggaccacggtcaccgtctcctcc

SEQ ID NO: 5 - protein, 111 aa, synthetic construct
  REGION 1..111: note = Synthetic
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 6 - DNA, 333 bp, synthetic construct
  source 1..333: mol_type = other DNA
  misc_feature 1..333: note = Synthetic
gatatccagctgacccagtctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctcgagatcaaa

SEQ ID NO: 7 - protein, 119 aa, synthetic construct
  REGION 1..119: note = Synthetic
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 8 - DNA, 357 bp, synthetic construct
  source 1..357: mol_type = other DNA
  misc_feature 1..357: note = Synthetic
gatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctca

SEQ ID NO: 9 - protein, 106 aa, synthetic construct
  REGION 1..106: note = Synthetic
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 10 - DNA, 318 bp, synthetic construct
  source 1..318: mol_type = other DNA
  misc_feature 1..318: note = Synthetic
gacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 11 - protein, 10 aa, synthetic construct
  REGION 1..10: note = Synthetic
GYTFTRYTMH

SEQ ID NO: 12 - protein, 17 aa, synthetic construct
  REGION 1..17: note = Synthetic
YINPSRGYTNYNQKFKD

SEQ ID NO: 13 - protein, 10 aa, synthetic construct
  REGION 1..10: note = Synthetic
YYDDHYCLDY

SEQ ID NO: 14 - protein, 10 aa, synthetic construct
  REGION 1..10: note = Synthetic
RASSSVSYMN

SEQ ID NO: 15 - protein, 7 aa, synthetic construct
  REGION 1..7: note = Synthetic
DTSKVAS

SEQ ID NO: 16 - protein, 9 aa, synthetic construct
  REGION 1..9: note = Synthetic
QQWSSNPLT

SEQ ID NO: 17 - protein, 10 aa, synthetic construct
  REGION 1..10: note = Synthetic
GYAFSSYWMN

SEQ ID NO: 18 - protein, 17 aa, synthetic construct
  REGION 1..17: note = Synthetic
QIWPGDGDTNYNGKFKG

SEQ ID NO: 19 - protein, 15 aa, synthetic construct
  REGION 1..15: note = Synthetic
RETTTVGRYYYAMDY

SEQ ID NO: 20 - protein, 15 aa, synthetic construct
  REGION 1..15: note = Synthetic
KASQSVDYDGDSYLN

SEQ ID NO: 21 - protein, 7 aa, synthetic construct
  REGION 1..7: note = Synthetic
DASNLVS

SEQ ID NO: 22 - protein, 9 aa, synthetic construct
  REGION 1..9: note = Synthetic
QQSTEDPWT